(19) United States
(12) Patent Application Publication
(10) Pub. No.: US 2003/0229128 A1
(43) Pub. Date: Dec. 11, 2003

(54) Glucagon receptor antagonists/inverse agonists

(76) Inventors: Peter Madsen, Bagsvaerd (DK); Carsten Behrens, Kobenhavn N. (DK)

Correspondence Address: Reza Green, Esq., Novo Nordisk Pharmaceuticals, Inc., 100 College Road West, Princeton, NJ, 08540, US

(21) Appl. No.: 10/322,168

(22) Filed: Dec. 18, 2002

Related U.S. Application Data
(60) Provisional application No. 60/342,428, filed on Dec. 20, 2001.

(30) Foreign Application Priority Data
Dec. 19, 2001 (DK) PA 2001 01917

(51) Int. Cl. C07D 249/16; C 07D 4 3/02; A61K 31/4184; A61K 31/198; A61K 31/353; A61K 31/4035

(52) U.S. Cl. 514/381; 514/563; 548/257; 562/439; 514/384; 514/417; 514/452; 548/309.1; 548/477; 549/355

(57) ABSTRACT

Novel compounds, which act to antagonize the action of the glucagon hormone on the glucagon receptor. Owing to their antagonizing effect of the glucagon receptor the compounds may be suitable for the treatment of any diseases and disorders, wherein a glucagon antagonistic action is beneficial, such as hyperglycemia, type 1 diabetes, type 2 diabetes, disorders of the lipid metabolism and obesity.

### CROSS REFERENCE TO RELATED APPLICATIONS

The present application claims the benefit under 35 U.S.C. 119 of Danish application PA 2001 01917, filed Dec. 19, 2001, and of U.S. provisional application No. 60/342,428, filed Dec. 20, 2001, the contents of both of which are hereby incorporated by reference.

### FIELD OF THE INVENTION

The present invention relates to agents that act to antagonize the action of the glucagon peptide hormone on the glucagon receptor. More particularly, it relates to glucagon antagonists or inverse agonists.

### BACKGROUND OF THE INVENTION

Glucagon is a key hormonal agent that, in co-operation with insulin, mediates homeostatic regulation of the amount of glucose in the blood. Glucagon primarily acts by stimulating certain cells (mostly liver cells) to release glucose when blood glucose levels fall. The action of glucagon is opposite to that of insulin, which stimulates cells to take up and store glucose whenever blood glucose levels rise. Both glucagon and insulin are peptide hormones.

Glucagon is produced in the alpha islet cells of the pancreas and insulin in the beta islet cells. Diabetes mellitus is a common disorder of glucose metabolism. The disease is characterized by hyperglycemia and may be classified as type 1 diabetes, the insulin-dependent form, or type 2 diabetes, which is non-insulin-dependent in character. Subjects with type 1 diabetes are hyperglycemic and hypoinsulinemic, and the conventional treatment for this form of the disease is to provide insulin. However, in some patients with type 1 or type 2 diabetes, absolute or relative elevated glucagon levels have been shown to contribute to the hyperglycemic state. Both in healthy control animals as well as in animal models of type 1 and type 2 diabetes, removal of circulating glucagon with selective and specific anti-bodies has resulted in reduction of the glycemic level. These studies suggest that glucagon suppression or an action that antagonizes glucagon could be a useful adjunct to conventional treatment of hyperglycemia in diabetic patients. The action of glucagon can be suppressed by providing an antagonist or an inverse agonist, ie substances that inhibit or prevent glucagon-induced responses. The antagonist can be peptidic or non-peptidic in nature.

Native glucagon is a 29 amino acid peptide having the sequence: His-Ser-Glu-Gly-Thr-Phe-Thr-Ser-Asp-Tyr-Ser-Lys-Tyr-Leu-Asp-Ser-Arg-Arg-Ala-Gln-Asp-Phe-Val-Gln-Trp-Leu-Met-Asn-Thr-OH

Glucagon exerts its action by binding to and activating its receptor, which is part of the Glucagon-Secretin branch of the 7-transmembrane G-protein coupled receptor family. The receptor functions by activating the adenylyl cyclase second messenger system and the result is an increase in cAMP levels.

Several publications disclose peptides that are stated to act as glucagon antagonists. Probably, the most thoroughly characterized antagonist is DesHis ¹[Glu⁹]-glucagon amide (Unson et al., Peptides 10, 1171 (1989); Post et al., Proc. Natl. Acad. Sci. USA 90, 1662 (1993)). Other antagonists are DesHis¹,Phe⁶[Glu⁹]-glucagon amide (Azizh et al., Bioorganic & Medicinal Chem. Lett. 16, 1849 (1995)) and NLeu⁹,Ala^{11,16}-glucagon amide (Unson et al., J. Biol. Chem. 269 (17), 12548 (1994)).

Peptide antagonists of peptide hormones are often quite potent. However, they are generally known not to be orally available because of degradation by physiological enzymes, and poor distribution in vivo. Therefore, orally available non-peptide antagonists of peptide hormones are generally preferred. Among the non-peptide glucagon antagonists, a quinoxaline derivative, (2-styryl-3-[3-(dimethylamino)propylmethylamino]-6,7-dichloroquinoxaline was found to displace glucagon from the rat liver receptor (Collins, J. L. et al., Bioorganic and Medicinal Chemistry Letters 2(9):915-918 (1992)). WO 94/14426 (The Wellcome Foundation Limited) discloses use of skyrin, a natural product comprising a pair of linked 9,10-anthracenedione groups, and its synthetic analogues, as glucagon antagonists. U.S. Pat. No. 4,359,474 (Sandoz) discloses the glucagon inhibiting properties of 1-phenyl pyrazole derivatives. U.S. Pat. No. 4,374,130 (Sandoz) discloses substituted disilacyclohexanes as glucagon inhibiting agents. WO 98/04528 (Bayer Corporation) discloses substituted pyridines and biphenyls as glucagon antagonists. U.S. Pat. No. 5,776,954 (Merck & Co., Inc.) discloses substituted pyridyl pyrroles as glucagon antagonists and WO 98/21957, WO 98/22108, WO 98/22109 and U.S. Pat. No. 5,880,139 (Merck & Co., Inc.) disclose 2,4-diaryl-5-pyridylimidazoles as glucagon antagonists. Furthermore, WO 97/16442 and U.S. Pat. No. 5,837,719 (Merck & Co., Inc.) disclose 2,5-substituted aryl pyrroles as glucagon antagonists. WO 98/24780, WO 98/24782, WO 99/24404 and WO 99/32448 (Amgen Inc.) disclose substituted pyrimidinone and pyridone compounds and substituted pyrimidine compounds, respectively, which are stated to possess glucagon antagonistic activity. Madsen et al. (J. Med. Chem. 1998 (41) 5151-7) discloses a series of 2-(benzimidazol-2-ylthio)-1-(3,4-dihydroxyphenyl)-1-ethanones as competitive human glucagon receptor antagonists. WO 99/01423 and WO 00/39088 (Novo Nordisk A/S) disclose different series of alkylidene hydrazides as glucagon antagonists/inverse agonists. WO 00/69810, WO 02/00612, WO 02/40444, WO 02/40445, WO 02/40446 (Novo Nordisk A/S) discloses further classes of glucagon antagonists.

These known glucagon antagonists differ structurally from the present compounds.

Definitions

The following is a detailed definition of the terms used to describe the compounds of the invention:

“Halogen” designates an atom selected from the group consisting of F, Cl, Br and I.

The term “C₁₋₆-alkyl” as used herein represents a saturated, branched or straight hydrocarbon group having from 1 to 6 carbon atoms. Representative examples include, but are not limited to, methyl, ethyl, n-propyl, isopropyl, butyl, isobutyl, sec-butyl, tert-butyl, n-pentyl, isopentyl, neopentyl, tert-pentyl, n-hexyl, isohexyl and the like.

The term “C₁₋₆alkoxy” as used herein refers to the radical —O—C₁₋₆-alkyl, wherein C₁₋₆-alkyl is as defined above. Representative examples are methoxy, ethoxy, n-propoxy, isopropoxy, butoxy, sec-butoxy, tert-butoxy, pentoxy, isopentoxy, hexoxy, isohexoxy and the like.

The term “C₃₋₈-cycloalkyl” as used herein represents a saturated, carbocyclic group having from 3 to 8 carbon atoms. Representative examples are cyclopropyl, cyclobutyl, cyclopentyl, cyclohexyl, cycloheptyl, cyclooctyl and the like.

The term “aryl” as used herein is intended to include carbocyclic, aromatic ring systems such as 6 membered monocyclic and 9 to 14 membered bi- and tricyclic, carbocyclic, aromatic ring systems. Representative examples are phenyl, biphenylyl, naphthyl, anthracenyl, phenanthrenyl, fluorenyl, indenyl, azulenyl and the like. Aryl is also intended to include the partially hydrogenated derivatives of the ring systems enumerated above. Non-limiting examples of such partially hydrogenated derivatives are 1,2,3,4-tetrahydronaphthyl, 1,4-dihydronaphthyl and the like.

The term “heteroaryl” as used herein is intended to include aromatic, heterocyclic ring systems containing one or more heteroatoms selected from nitrogen, oxygen and sulfur such as 5 to 7 membered monocyclic or 8 to 14 membered bi- and tricyclic aromatic, heterocyclic ring systems containing one or more heteroatoms selected from nitrogen, oxygen and sulfur. Representative examples are furyl, thienyl, pyrrolyl, oxazolyl, thiazolyl, imidazolyl, isoxazolyl, isothiazolyl, 1,2,3-triazolyl, 1,2,4-triazolyl, pyranyl, pyridyl, pyridazinyl, pyrimidinyl, pyrazinyl, 1,2,3-triazinyl, 1,2,4-triazinyl, 1,3,5- triazinyl, 1,2,3-oxadiazolyl, 1,2,4-oxadiazolyl, 1,2,5-oxadiazolyl, 1,3,4-oxadiazolyl, 1,2,3-thiadiazolyl, 1,2,4-thiadiazolyl, 1,2,5-thiadiazolyl, 1,3,4-thiadiazolyl, tetrazolyl, thiadiazinyl, indolyl, isoindolyl, benzofuryl, benzothienyl, indazolyl, benzimidazolyl, benzthiazolyl, benzisothiazolyl, benzoxazolyl, benzisoxazolyl, purinyl, quinazolinyl, quinolizinyl, quinolinyl, isoquinolinyl, quinoxalinyl, naphthyridinyl, pteridinyl, carbazolyl, azepinyl, diazepinyl, acridinyl and the like. Heteroaryl is also intended to include the partially hydrogenated derivatives of the ring systems enumerated above. Non-limiting examples of such partially hydrogenated derivatives are 2,3-dihydrobenzofuranyl, pyrrolinyl, pyrazolinyl, indolinyl, oxazolidinyl, oxazolinyl, oxazepinyl and the like.

“Aryl-C₁₋₆-alkyl”, “heteroaryl-C₁₋₆alkyl”, etc. mean C₁₋₆alkyl as defined above, substituted by an aryl or heteroaryl as defined above, for example:

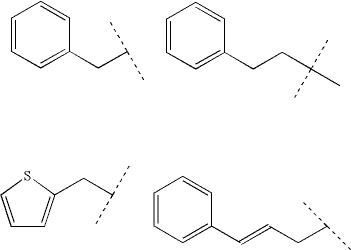

The term “optionally substituted” as used herein means that the groups in question are either unsubstituted or substituted with one or more of the substituents specified. When the groups in question are substituted with more than one substituent the substituents may be the same or different.

Certain of the above defined terms may occur more than once in the structural formulae, and upon such occurrence each term shall be defined independently of the other.

Furthermore, when using the terms “independently are” and “independently selected from” it should be understood that the groups in question may be the same or different.

The term “treatment” as used herein means the management and care of a patient for the purpose of combating a disease, disorder or condition. The term is intended to include the delaying of the progression of the disease, disorder or condition, the alleviation or relief of symptoms and complications, and/or the cure or elimination of the disease, disorder or condition. The patient to be treated is preferably a mammal, in particular a human being.

### DESCRIPTION OF THE INVENTION

The present invention is based on the unexpected observation that the compounds of the general formula (I) disclosed below show a high binding affinity for the glucagon receptor and antagonize the action of glucagon.

Accordingly, the invention relates to a compound of the formula (I):

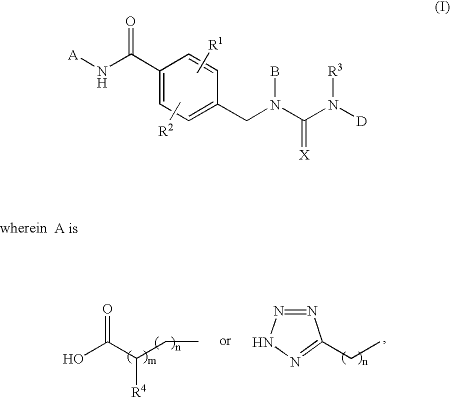

m is 0 or 1,

n is 0, 1, 2 or 3,

with the proviso that m and n must not be 0 at the same time,

R⁴ is hydrogen, fluoro or —(CH₂)ₚ—OR⁵,

p is 0 or 1,

R⁵ is hydrogen, C₁₋₆-alkyl, C₁₋₆-alkanoyl, aryl or aryl-C₁₋₆-alkyl,

R¹ and R² independently are hydrogen, halogen, trifluoromethyl, trifluoromethoxy, cyano, trifluoromethylthio, nitro, C₁₋₆-alkyl, C₁₋₆alkoxy, hydroxy, C₁₋₆-alkylthio, C₁₋₆-alkylsulfonyl, trifluoromethylsulfonyl or —NR⁶R⁷,

R⁶ and R⁷ independently are hydrogen or C₁₋₆-alkyl,

B is

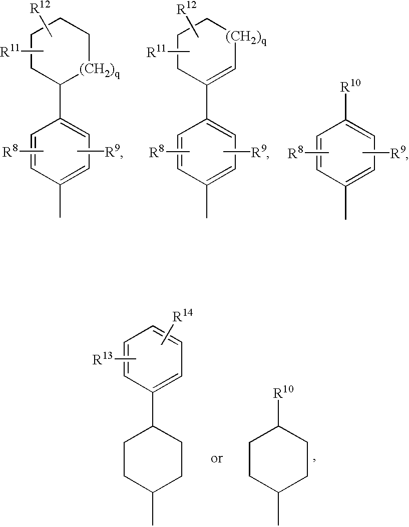

R⁸, R⁹, R¹³ and R¹⁴ independently are hydrogen, halogen, trifluoromethyl, C₁₋₆-alkyl or C₁₋₆-alkoxy,

R¹⁰ is hydrogen, halogen, trifluoromethyl, trifluoromethoxy, cyano, trifluoromethylthio, nitro, C₁₋₆-alkyl, methylthio or C₃₋₈-cycloalkyl,

R¹¹ and R¹² independently are hydrogen or C₁₋₆-alkyl,

q is 0, 1, 2 or 3;

R³ is hydrogen or C₁₋₈-alkyl,

X is ═N—CN, ═N—CH₂R¹⁵, ═CH—NO₂ or ═CHR¹⁵,

R¹⁵ is

hydrogen, cyano or trifluoromethyl,

C₁₋₆-alkyl, which may optionally be substituted with

fluoro, trifluoromethyl, trifluoromethoxy, cyano, trifluoromethylthio, C₁₋₆-alkoxy, hydroxy, C₁₋₆-alkylthio, C₁₋₆-alkylsulfonyl, trifluoromethylsulfonyl or —NR¹⁶R¹⁷,

R¹⁶ and R¹⁷ independenly are hydrogen or C₁₋₆-alkyl,

aryl or heteroaryl, which may optionally be substituted with

halogen, trifluoromethyl, trifluoromethoxy, cyano, trifluoromethylthio, nitro, C₁₋₆-alkyl, C₁₋₆-alkoxy, hydroxy, C₁₋₆-alkylthio, C₁₋₆-alkylsulfonyl, trifluoromethylsulfonyl or —NR¹⁸R¹⁹,

R¹⁸ and R¹⁹ independenly are hydrogen or C₁₋₆-alkyl,

D is

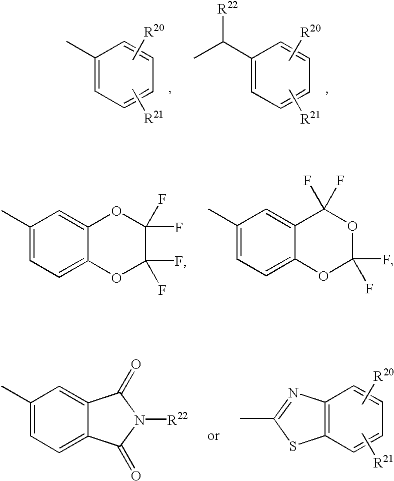

R²⁰ and R²¹ independently are hydrogen, halogen, trifluoromethyl, trifluoromethoxy, cyano, trifluoromethylthio, nitro, C₁₋₆-alkyl, aryl, methylthio, methylsulfonyl, trifluoromethylsulfonyl, —NR²³R²⁴, C₃₋₈-cycloalkyl or —S(O)₂—N(C₁₋₆-alkyl)(aryl),

R²³ and R²⁴ independently are hydrogen or C₁₋₆-alkyl,

R²² is hydrogen, C₁₋₆-alkyl, C₃₋₈-cycloalkyl or C₃₋₈-cycloalkyl-C₁₋₆-alkyl,

as well as any diastereomer or enantiomer or tautomeric form thereof including mixtures of these or a pharmaceutically acceptable salt thereof.

In one embodiment A is

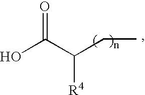

wherein n and R⁴ is as defined for formula (I), such as

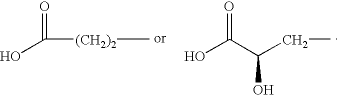

In another embodiment A is

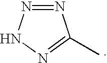

In yet another embodiment R¹ and R² are both hydrogen.

In still another embodiment B is

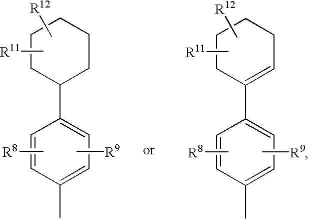

wherein R⁸, R⁹, R¹¹ and R¹² are as defined for formula (I).

In one embodiment R⁸ and R⁹ are both hydrogen.

In another embodiment R¹¹ and R¹² are both hydrogen.

In still a further embodiment B is

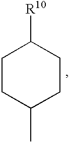

wherein R¹⁰ is as defined for formula (I), such as C₁₋₆-alkyl.

In another embodiment X is ═N—CN, ═CH—NO₂, ═N—CH₂—CF₃ or ═N—CH₂—CN.

In still another embodiment R³ is hydrogen.

In yet another embodiment D is

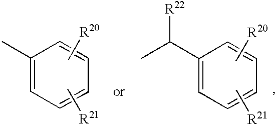

wherein R²⁰, R²¹ and R²² are as defined for formula (I).

In one embodiment R²⁰ and R²¹ are independently hydrogen, halogen, trifluoromethyl or trifluoromethoxy, and R²² is C₁₋₆-alkyl.

The compounds of the present invention may be chiral, and it is intended that any enantiomers, as separated, pure or partially purified enantiomers or racemic mixtures thereof are included within the scope of the invention.

Furthermore, when a double bond or a fully or partially saturated ring system or more than one center of asymmetry or a bond with restricted rotatability is present in the molecule diastereomers may be formed. It is intended that any diastereomers, as separated, pure or partially purified diastereomers or mixtures thereof are included within the scope of the invention.

Furthermore, some of the compounds of the present invention may exist in different tautomeric forms and it is intended that any tautomeric forms, which the compounds are able to form, are included within the scope of the present invention.

The present invention also encompasses pharmaceutically acceptable salts of the present compounds. Such salts include pharmaceutically acceptable acid addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedi-sulfonic, gluconic, citraconic, aspartic, stearic, palmitic, EDTA, glycolic, p-aminobenzoic, glutamic, benzenesulfonic, p-toluenesulfonic acids and the like. Further examples of pharmaceutically acceptable inorganic or organic acid addition salts include the pharmaceutically acceptable salts listed in J. Pharm. Sci. 1977, 66, 2, which is incorporated herein by reference. Examples of metal salts include lithium, sodium, potassium, magnesium salts and the like. Examples of ammonium and alkylated ammonium salts include ammonium, methyl-, dimethyl-, trimethyl-, ethyl-, hydroxyethyl-, diethyl-, butyl-, tetramethylammonium salts and the like.

Also intended as pharmaceutically acceptable acid addition salts are the hydrates, which the present compounds, are able to form.

Furthermore, the pharmaceutically acceptable salts comprise basic amino acid salts such as lysine, arginine and ornithine.

The acid addition salts may be obtained as the direct products of compound synthesis. In the alternative, the free base may be dissolved in a suitable solvent containing the appropriate acid, and the salt isolated by evaporating the solvent or otherwise separating the salt and solvent.

The compounds of the present invention may form solvates with standard low molecular weight solvents using methods well known to the person skilled in the art. Such solvates are also contemplated as being within the scope of the present invention.

The invention also encompasses prodrugs of the present compounds, which on administration undergo chemical conversion by metabolic processes before becoming pharmacologically active substances. In general, such prodrugs will be functional derivatives of present compounds, which are readily convertible in vivo into the required compound. Conventional procedures for the selection and preparation of suitable prodrug derivatives are described, for example, in “Design of Prodrugs”, ed. H. Bundgaard, Elsevier, 1985.

The invention also encompasses active metabolites of the present compounds.

The compounds according to the present invention act to antagonize the action of glucagon and are accordingly useful for the treatment of disorders and diseases in which such an antagonism is beneficial.

The compounds according to the invention preferably have an IC₅₀ value of no greater than 5 μM as determined by the Glucagon Binding Assay (I) or Glucagon Binding Assay (II) disclosed herein.

More preferably, the compounds according to the invention have an IC₅₀ value of less than 1 μM, preferably of less than 500 nM and even more preferred of less than 100 nM as determined by the Glucagon Binding Assay (I) or Glucagon Binding Assay (II) disclosed herein.

Furthermore, the compounds according to the invention preferably have a higher binding affinity to the glucagon receptor than to the GIP receptor.

Accordingly, the present compounds may be applicable for the treatment of hyperglycemia, IGT (impaired glucose tolerance), insulin resistance syndromes, syndrome X, type 1 diabetes, type 2 diabetes, hyperlipidemia, dyslipidemia, hypertriglyceridemia, hyperlipoproteinemia, hypercholesterolemia, arteriosclerosis including atherosclerosis, glucagonomas, acute pancreatitis, cardiovascular diseases, hypertension, cardiac hypertrophy, gastrointestinal disorders, obesity, diabetes as a consequence of obesity, diabetic dyslipidemia, etc.

Furthermore, they may be applicable as diagnostic agents for identifying patients having a defect in the glucagon receptor, as a therapy to increase gastric acid secretions and to reverse intestinal hypomobility due to glucagon administration.

They may also be useful as tool or reference molecules in labelled form in binding assays to identify new glucagon antagonists.

Accordingly, in a further aspect the invention relates to a compound according to the invention for use as a medicament.

The invention also relates to pharmaceutical compositions comprising, as an active ingredient, at least one compound according to the invention together with one or more pharmaceutically acceptable carriers or excipients.

The pharmaceutical composition is preferably in unit dosage form, comprising from about 0.05 mg to about 1000 mg, preferably from about 0.1 mg to about 500 mg and especially preferred from about 0.5 mg to about 200 mg of the compound according to the invention.

Furthermore, the invention relates to the use of a compound according to the invention for the preparation of a pharmaceutical composition for the treatment of a disorder or disease, wherein a glucagon antagonistic action is beneficial.

The invention also relates to a method for the treatment of disorders or diseases, wherein a glucagon antagonistic action is beneficial the method comprising administering to a subject in need thereof an effective amount of a compound according to the invention.

In one embodiment of the invention the present compounds are used for the preparation of a medicament for the treatment of any glucagon-mediated conditions and diseases.

In another embodiment of the invention the present compounds are used for the preparation of a medicament for the treatment of hyperglycemia.

In yet another embodiment of the invention the present compounds are used for the preparation of a medicament for lowering blood glucose in a mammal. The present compounds are effective in lowering the blood glucose, both in the fasting and the postprandial stage.

In still another embodiment of the invention the present compounds are used for the preparation of a pharmaceutical composition for the treatment of IGT.

In a further embodiment of the invention the present compounds are used for the preparation of a pharmaceutical composition for the treatment of type 2 diabetes.

In yet a further embodiment of the invention the present compounds are used for the preparation of a pharmaceutical composition for the delaying or prevention of the progression from IGT to type 2 diabetes.

In yet another embodiment of the invention the present compounds are used for the preparation of a pharmaceutical composition for the delaying or prevention of the progression from non-insulin requiring type 2 diabetes to insulin requiring type 2 diabetes.

In a further embodiment of the invention the present compounds are used for the preparation of a pharmaceutical composition for the treatment of type 1 diabetes. Such treatment is normally accompanied by insulin therapy.

In yet a further embodiment of the invention the present compounds are used for the preparation of a pharmaceutical composition for the treatment of obesity.

In still a further embodiment of the invention the present compounds are used for the preparation of a pharmaceutical composition for the treatment of disorders of the lipid metabolism.

In still another embodiment of the invention the present compounds are used for the preparation of a pharmaceutical composition for the treatment of an appetite regulation or energy expenditure disorder.

In a further embodiment of the invention, treatment of a patient with the present compounds is combined with diet and/or exercise.

In a further aspect of the invention the present compounds are administered in combination with one or more further active substances in any suitable ratios. Such further active substances may eg be selected from antidiabetics, antiobesity agents, antihypertensive agents, agents for the treatment of complications resulting from or associated with diabetes and agents for the treatment of complications and disorders resulting from or associated with obesity.

Thus, in a further embodiment of the invention the present compounds may be administered in combination with one or more antidiabetics.

Suitable antidiabetic agents include insulin, insulin analogues and derivatives such as those disclosed in EP 792 290 (Novo Nordisk A/S), eg N^{εB29}-tetradecanoyl des (B30) human insulin, EP 214 826 and EP 705 275 (Novo Nordisk A/S), eg Asp^{B28} human insulin, U.S. Pat. No. 5,504,188 (Eli Lilly), eg LyS^{B28} Pro^{B29} human insulin, EP 368 187 (Aventis), eg Lantus®, which are all incorporated herein by reference, GLP-1 and GLP-1 derivatives such as those disclosed in WO 98/08871 (Novo Nordisk A/S), which is incorporated herein by reference, as well as orally active hypoglycemic agents.

The orally active hypoglycemic agents preferably comprise imidazolines, sulphonylureas, biguanides, meglitinides, oxadiazolidinediones, thiazolidinediones, insulin sensitizers, insulin secretagogues, such as glimepride, α-glucosidase inhibitors, agents acting on the ATP-dependent potassium channel of the β-cells eg potassium channel openers such as those disclosed in WO 97/26265, WO 99/03861 and WO 00/37474 (Novo Nordisk A/S) which are incorporated herein by reference, or mitiglinide, or a potassium channel blocker, such as BTS-67582, nateglinide, glucagon antagonists such as those disclosed in WO 99/01423 and WO 00/39088 (Novo Nordisk A/S and Agouron Pharmaceuticals, Inc.), which are incorporated herein by reference, GLP-1 agonists such as those disclosed in WO 00/42026 (Novo Nordisk A/S and Agouron Pharmaceuticals, Inc.), which are incorporated herein by reference, DPP-IV (dipeptidyl peptidase-IV) inhibitors, PTPase (protein tyrosine phosphatase) inhibitors, inhibitors of hepatic enzymes involved in stimulation of gluconeogenesis and/or glycogenolysis, glucose uptake modulators, GSK-3 (glycogen synthase kinase-3) inhibitors, compounds modifying the lipid metabolism such as antilipidemic agents, compounds lowering food intake, PPAR (peroxisome proliferator-activated receptor) and RXR (retinoid X receptor) agonists, such as ALRT-268, LG-1268 or LG-1069.

In one embodiment, the present compounds are administered in combination with insulin or an insulin analogue or derivative, such as N^{εB29} -tetradecanoyl des (B30) human insulin, Asp^{B28} human insulin, LyS^{B28} Pro^{B29} human insulin, Lantus®, or a mix-preparation comprising one or more of these.

In a further embodiment of the invention the present compounds are administered in combination with a sulphonylurea eg tolbutamide, chlorpropamide, tolazamide, glibenclamide, glipizide, glimepiride, glicazide or glyburide.

In another embodiment of the invention the present compounds are administered in combination with a biguanide eg metformin.

In yet another embodiment of the invention the present compounds are administered in combination with a meglitinide eg repaglinide or nateglinide.

In still another embodiment of the invention the present compounds are administered in combination with a thiazolidinedione insulin sensitizer eg troglitazone, ciglitazone, pioglitazone, rosiglitazone, isaglitazone, darglitazone, englitazone, CS-011/Cl-1037 or T 174 or the compounds disclosed in WO 97/41097, WO 97/41 119, WO 97/41120, WO 00/41121 and WO 98/45292 (Dr. Reddy's Research Foundation), which are incorporated herein by reference.

In still another embodiment of the invention the present compounds may be administered in combination with an insulin sensitizer eg such as GI 262570, YM-440, MCC-555, JTT-501, AR-H039242, KRP-297, GW-409544, CRE-16336, AR-H049020, LY510929, MBX-102, CLX-0940, GW-501516 or the compounds disclosed in WO 99/19313, WO 00/50414, WO 00/63191, WO 00/63192, WO 00/63193 (Dr. Reddy's Research Foundation) and WO 00/23425, WO 00/23415, WO 00/23451, WO 00/23445, WO 00/23417, WO 00/23416, WO 00/63153, WO 00/63196, WO 00/63209, WO 00/63190 and WO 00/63189 (Novo Nordisk A/S), which are incorporated herein by reference.

In a further embodiment of the invention the present compounds are administered in combination with an a-glucosidase inhibitor eg voglibose, emiglitate, miglitol or acarbose.

In another embodiment of the invention the present compounds are administered in combination with an agent acting on the ATP-dependent potassium channel of the β-cells eg tolbutamide, glibenclamide, glipizide, glicazide, BTS-67582 or repaglinide.

In yet another embodiment of the invention the present compounds may be administered in combination with nateglinide.

In still another embodiment of the invention the present compounds are administered in combination with an antilipidemic agent eg cholestyramine, colestipol, clofibrate, gemfibrozil, lovastatin, pravastatin, simvastatin, probucol or dextrothyroxine.

In another embodiment of the invention, the present compounds are administered in combination with more than one of the above-mentioned compounds eg in combination with metformin and a sulphonylurea such as glyburide; a sulphonylurea and acarbose; nateglinide and melformin; acarbose and melformin; a sulfonylurea, metformin and troglitazone; insulin and a sulfonylurea; insulin and melformin; insulin, metformin and a sulfonylurea; insulin and troglitazone; insulin and lovastatin; etc.

In a further embodiment of the invention the present compounds may be administered in combination with one or more antiobesity agents or appetite regulating agents.

Such agents may be selected from the group consisting of CART (cocaine amphetamine regulated transcript) agonists, NPY (neuropeptide Y) antagonists, MC4 (melanocortin 4) agonists, MC3 (melanocortin 3) agonists, orexin antagonists, TNF (tumor necrosis factor) agonists, CRF (corticotropin releasing factor) agonists, CRF BP (corticotropin releasing factor binding protein) antagonists, urocortin agonists, β3 adrenergic agonists such as CL-316243, AJ-9677, GW-0604, LY362884, LY377267 or AZ-40140, MSH (melanocyte-stimulating hormone) agonists, MCH (melanocyte-concentrating hormone) antagonists, CCK (cholecystokinin) agonists, serotonin re-uptake inhibitors such as fluoxetine, seroxat or citalopram, serotonin and noradrenaline re-uptake inhibitors, mixed serotonin and noradrenergic compounds, 5HT (serotonin) agonists, bombesin agonists, galanin antagonists, growth hormone, growth factors such as prolactin or placental lactogen, growth hormone releasing compounds, TRH (thyreotropin releasing hormone) agonists, UCP 2 or 3 (uncoupling protein 2 or 3) modulators, leptin agonists, DA agonists (bromocriptin, doprexin), lipase/amylase inhibitors, PPAR (peroxisome proliferator-activated receptor) modulators, RXR (retinoid X receptor) modulators, TR β agonists, AGRP (Agouti related protein) inhibitors, H3 histamine antagonists, opioid antagonists (such as naltrexone), exendin-4, GLP-1 and ciliary neurotrophic factor.

In one embodiment of the invention the antiobesity agent is leptin.

In another embodiment the antiobesity agent is dexamphetamine or amphetamine.

In another embodiment the antiobesity agent is fenfluramine or dexfenfluramine.

In still another embodiment the antiobesity agent is sibutramine.

In a further embodiment the antiobesity agent is orlistat.

In another embodiment the antiobesity agent is mazindol or phentermine.

In still another embodiment the antiobesity agent is phendimetrazine, diethylpropion, fluoxetine, bupropion, topiramate or ecopipam.

Furthermore, the present compounds may be administered in combination with one or more antihypertensive agents. Examples of antihypertensive agents are β-blockers such as alprenolol, atenolol, timolol, pindolol, propranolol and metoprolol, ACE (angiotensin converting enzyme) inhibitors such as benazepril, captopril, enalapril, fosinopril, lisinopril, quinapril and ramipril, calcium channel blockers such as nifedipine, felodipine, nicardipine, isradipine, nimodipine, diltiazem and verapamil, and α-blockers such as doxazosin, urapidil, prazosin and terazosin. Further reference can be made to Remington: The Science and Practice of Pharmacy, 19^{th} Edition, Gennaro, Ed., Mack Publishing Co., Easton, Pa., 1995.

It should be understood that any suitable combination of the compounds according to the invention with diet and/or exercise, one or more of the above-mentioned compounds and optionally one or more other active substances are considered to be within the scope of the present invention.

Pharmaceutical Compositions

The compounds of the invention may be administered alone or in combination with pharma-ceutically acceptable carriers or excipients, in either single or multiple doses. The pharmaceu-tical compositions according to the invention may be formulated with pharmaceutically acceptable carriers or diluents as well as any other known adjuvants and excipients in accordance with conventional techniques such as those disclosed in Remington: The Science and Practice of Pharmacy, 19^{th} Edition, Gennaro, Ed., Mack Publishing Co., Easton, Pa., 1995.

The pharmaceutical compositions may be specifically formulated for administration by any suitable route such as the oral, rectal, nasal, pulmonary, topical (including buccal and sublingual), transdermal, intracisternal, intraperitoneal, vaginal and parenteral (including sub-cutaneous, intramuscular, intrathecal, intravenous and intradermal) route, the oral route being preferred. It will be appreciated that the preferred route will depend on the general condition and age of the subject to be treated, the nature of the condition to be treated and the active ingredient chosen.

Pharmaceutical compositions for oral administration include solid dosage forms such as capsules, tablets, dragees, pills, lozenges, powders and granules. Where appropriate, they can be prepared with coatings such as enteric coatings or they can be formulated so as to provide controlled release of the active ingredient such as sustained or prolonged release according to methods well known in the art.

Liquid dosage forms for oral administration include solutions, emulsions, suspensions, syrups and elixirs.

Pharmaceutical compositions for parenteral administration include sterile aqueous and non-aqueous injectable solutions, dispersions, suspensions or emulsions as well as sterile powders to be reconstituted in sterile injectable solutions or dispersions prior to use. Depot injectable formulations are also contemplated as being within the scope of the present invention.

Other suitable administration forms include suppositories, sprays, ointments, cremes, gels, inhalants, dermal patches, implants etc.

A typical oral dosage is in the range of from about 0.001 to about 100 mg/kg body weight per day, preferably from about 0.01 to about 50 mg/kg body weight per day, and more preferred from about 0.05 to about 10 mg/kg body weight per day administered in one or more dosages such as 1 to 3 dosages. The exact dosage will depend upon the frequency and mode of administration, the sex, age, weight and general condition of the subject treated, the nature and severity of the condition treated and any concomitant diseases to be treated and other factors evident to those skilled in the art.

The formulations may conveniently be presented in unit dosage form by methods known to those skilled in the art. A typical unit dosage form for oral administration one or more times per day such as 1 to 3 times per day may contain from 0.05 to about 1000 mg, preferably from about 0.1 to about 500 mg, and more preferred from about 0.5 mg to about 200 mg.

For parenteral routes such as intravenous, intrathecal, intramuscular and similar administration, typically doses are in the order of about half the dose employed for oral administration.

The compounds of this invention are generally utilized as the free substance or as a pharmaceutically acceptable salt thereof. One example is an acid addition salt of a compound having the utility of a free base. When a compound according to the invention contains a free base such salts are prepared in a conventional manner by treating a solution or suspension of a free base of the compound in question with a chemical equivalent of a pharmaceutically acceptable acid. Representative examples are mentioned above. Physiologically acceptable salts of a compound with a hydroxy group include the anion of said compound in combination with a suitable cation such as sodium or ammonium ion.

For parenteral administration, solutions of the novel compounds according to the invention in sterile aqueous solution, aqueous propylene glycol or sesame or peanut oil may be employed. Such aqueous solutions should be suitably buffered if necessary and the liquid diluent first rendered isotonic with sufficient saline or glucose. The aqueous solutions are particularly suitable for intravenous, intramuscular, subcutaneous and intraperitoneal administration. The sterile aqueous media employed are all readily available by standard techniques known to those skilled in the art.

Suitable pharmaceutical carriers include inert solid diluents or fillers, sterile aqueous solution and various organic solvents. Examples of solid carriers are lactose, terra alba, sucrose, cyclodextrin, talc, gelatine, agar, pectin, acacia, magnesium stearate, stearic acid and lower alkyl ethers of cellulose. Examples of liquid carriers are syrup, peanut oil, olive oil, phospholipids, fatty acids, fatty acid amines, polyoxyethylene and water. Similarly, the carrier or diluent may include any sustained release material known in the art, such as glyceryl monostearate or glyceryl distearate, alone or mixed with a wax. The pharmaceutical compositions formed by combining the present compounds and the pharmaceutically acceptable carriers are then readily administered in a variety of dosage forms suitable for the disclosed routes of administration. The formulations may conveniently be presented in unit dosage form by methods known in the art of pharmacy.

Formulations of the present invention suitable for oral administration may be presented as discrete units such as capsules or tablets, each containing a predetermined amount of the active ingredient, and which may include a suitable excipient. Furthermore, the orally available formulations may be in the form of a powder or granules, a solution or suspension in an aqueous or non-aqueous liquid, or an oil-in-water or water-in-oil liquid emulsion.

If a solid carrier is used for oral administration, the preparation may be tabletted, placed in a hard gelatine capsule in powder or pellet form or it can be in the form of a troche or lozenge. The amount of solid carrier will vary widely but will usually be from about 25 mg to about 1 g. If a liquid carrier is used, the preparation may be in the form of a syrup, emulsion, soft gelatine capsule or sterile injectable liquid such as an aqueous or non-aqueous liquid suspension or solution.

A typical tablet that may be prepared by conventional tabletting techniques may contain:

| | | |
|---|---|---|
| Core: | | |
| Active compound (as free compound or salt thereof) | | 5.0 mg |
| Lactosum Ph. Eur. | | 67.8 mg |
| Cellulose, microcryst. (Avicel) | | 31.4 mg |
| Amberlite ® IRP88* | | 1.0 mg |
| Magnesii stearas Ph. Eur. | | q.s. |
| Coating: | | |
| Hydroxypropyl methylcellulose | approx. | 9 mg |
| Mywacett 9-40 T** | approx. | 0.9 mg |

Polacrillin potassium NF, tablet disintegrant, Rohm and Haas.
**Acylated monoglyceride used as plasticizer for film coating.

If desired, the pharmaceutical composition of the invention may comprise a compound according to the invention in combination with further pharmacologically active substances such as those described in the foregoing.

### EXAMPLES

The compounds according to the invention may be prepared according to the following general procedures. The chemical reactions described are disclosed in terms of their general applicability to the preparation of the glucagon antagonists of the invention. Occasionally, the reaction may not be applicable as described to each compound included within the disclosed scope of the invention. The compounds for which this occurs will be readily recognised by those skilled in the art. In these cases the reactions can be successfully performed by conventional modifications known to those skilled in the art, that is, by appropriate protection of interfering groups, by changing to other conventional reagents, or by routine modification of reaction conditions. Alternatively, other reactions disclosed herein or otherwise conventional will be applicable to the preparation of the corresponding compounds of the invention. In all preparative methods, all starting materials are known or may easily be prepared from known starting materials.

General Procedure (A) for Solution Phase Preparation of Compounds of General Formula (I₁):

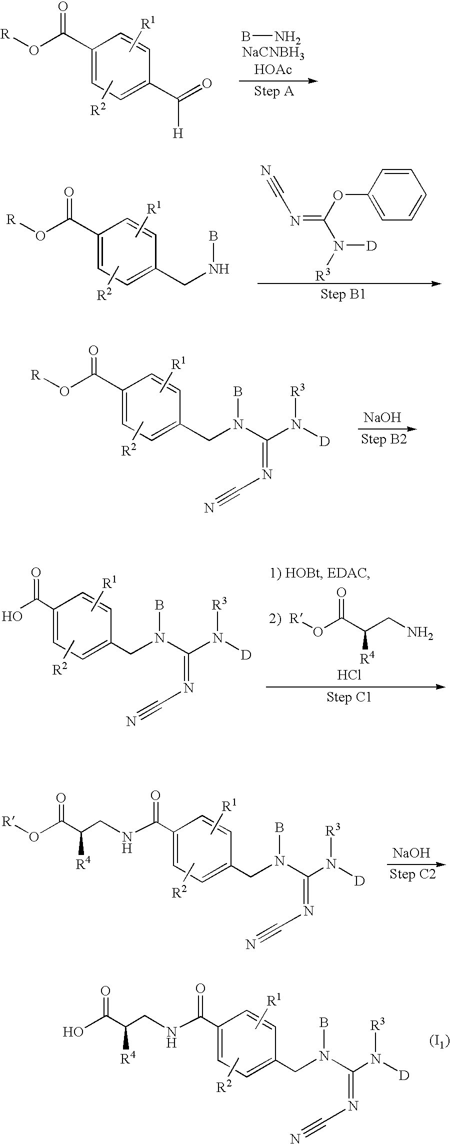

wherein R¹, R², R³, R⁴, B and D are as defined for formula (I), R and R′ are protecting groups such as C₁₋₆-alkyl and may be the same or different.

Step A is a reductive amination of the protected 4-formylbenzoic acid with B—NH₂ and a reducing agent such as sodium cyanoborohydride.

Step B1 is performed as described in K. S. Atwal et al., Tet. Lett., 1994, 8085-8088, by reacting the intermediate from step A with a N-cyano-O-phenylisourea (which in turn can be prepared from NHR³-D with diphenylcyanocarbonimidate in acetonitrile in the presence of sodium hydride) promoted by trimethylaluminum in dichloroethane.

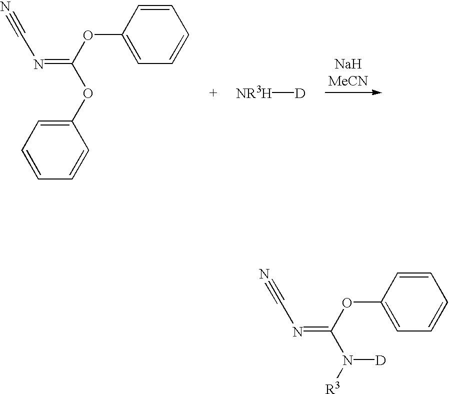

Step B2 is a deprotection (hydrolysis) of the benzoic acid.

Step C1 is a coupling reaction (amide bond formation) of the benzoic acid intermediate from step B2 with a 3-aminopropionic ester derivative.

Step C2 is a hydrolysis of the 3-aminopropionic acid derivative from step C1 to give the compounds of general formula (I₁) of the invention.

All steps except from step B1 are in analogy with similar transformations described in WO 00/69810.

General Procedure (B) for Solution Phase Preparation of Compounds of General Formula (I₁):

The order of reactions as compared to general procedure (A) may be changed so that the product from step A is reacted with diphenylcyanocarbonimidate (step B1) and the resulting N-cyano-O-phenylisourea is reacted with NHR³-D promoted by trimethylaluminum in dichloroethane (step B2). Otherwise the steps are as described for the corresponding steps in general procedure (A).

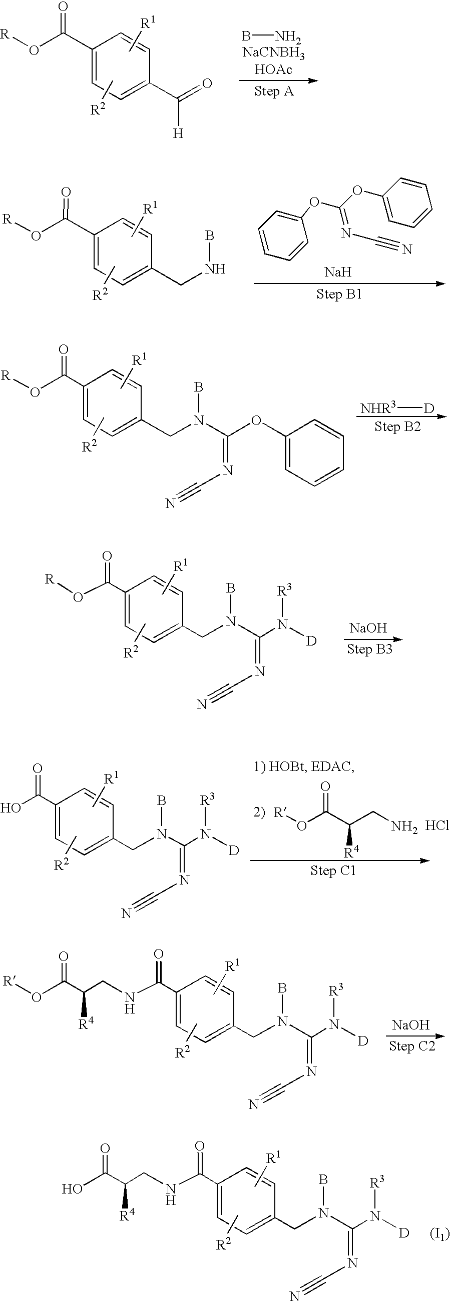

wherein R¹, R², R³, R⁴, B and D are as defined for formula (I), R and R¹ are protecting groups such as C₁₋₆-alkyl and may be the same or different.

General Procedure (C) for Solution Phase Preparation of Compounds of General Formula (I₁):

Alternatively, step B1 of general procedure (A) may be modified so that the product from step A is reacted with N-cyanothioureas and carbodiimides, eg EDAC (3-ethyl-1-(3-di-methylaminopropyl)carbodiimide). The N-cyanothioureas are in turn prepared from isothiocyanates and sodium cyanamide as described in K. S. Atwal et al., Tet. Lett., 1989, 7313-7316.

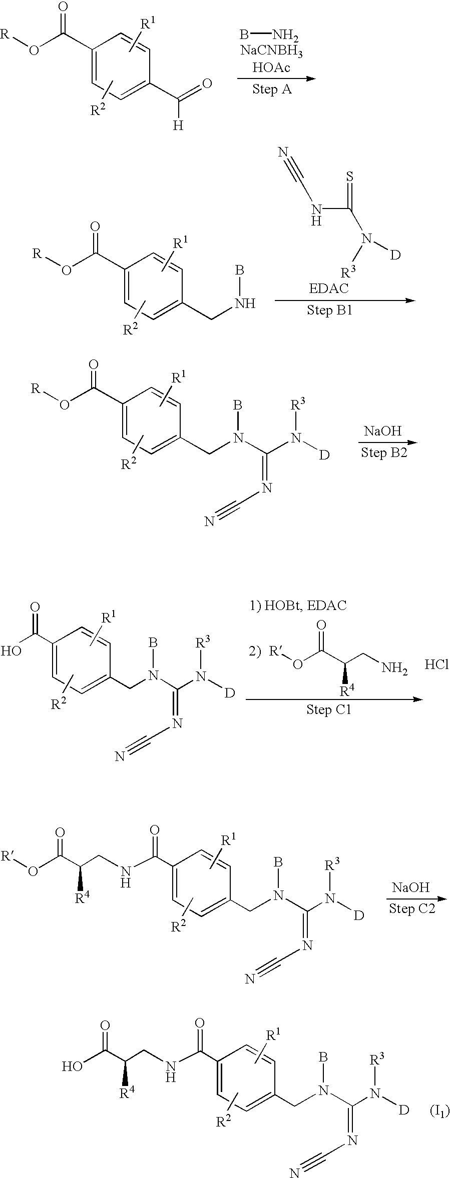

wherein R¹, R², R³, R⁴, B and D are as defined for formula (I), R and R′ are protecting groups such as C₁₋₆-alkyl and may be the same or different.

General Procedure (D) for Solution Phase Preparation of Compounds of General Formula (I₂):

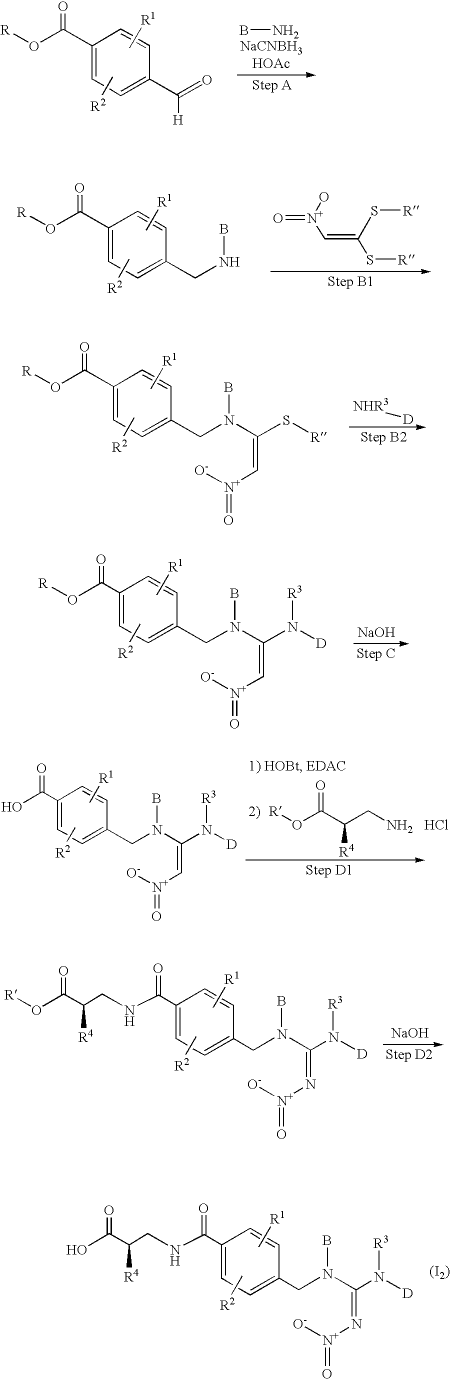

wherein R¹, R², R³, R⁴, B and D are as defined for formula (I), R, R′ and R″ are protecting groups such as C₁₋₆-alkyl and may be the same or different.

Step A is as described in step A of general procedure (A).

Step B1 is carried out in a similar way as described in D. M. Argilagos et al., Helv. Chim Acta, 1997, 273-292, by reacting the intermediate from step A with commercially available 1,1-bis(methylthio)-2-nitroethylene followed by reaction with D-NHR³ (Step B2).

Step C is a deprotection (hydrolysis) of the benzoic acid ester obtained in step B2.

Step D1 is a coupling reaction (amide bond formation) of the benzoic acid intermediate from step C with a 3-aminopropionic acid ester derivative.

Step D2 is a hydrolysis of the 3-aminopropionic acid derivative from step D1 to give the compounds of general formula (I₂) of the invention.

All steps except steps B1 and B2 are in analogy with similar transformations described in WO 00/69810.

General Procedure (E) for Solution Phase Preparation of Compounds of General Formula (I₂):

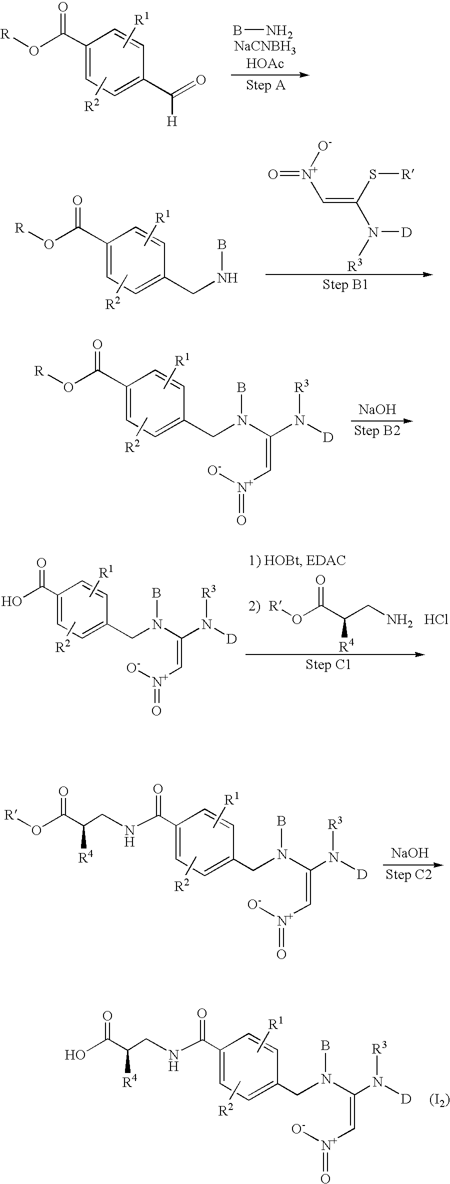

wherein R¹, R², R³, R⁴, B and D are as defined for formula (I), R and R′ are protecting groups such as C₁₋₆-alkyl and may be the same or different.

Step A is as described in step A in general procedure (A).

Step B1 is carried out in a similar way as described in D. M. Argilagos et al.: Helv. Chim Acta, 1997, 273-292, and in Schaefer et al.: J. Prakt. Chem., 1977, Vol 319, 149-157 by reacting the intermediate from step A with the substituted (1-methylsulfanyl-2-nitrovinyl)-amine which in turn may be prepared from 1,1-bis(methylthio)-2-nitroethylene with D-NHR³ or from the reaction of isothiocyanates with nitromethane followed by alkylation as described below:

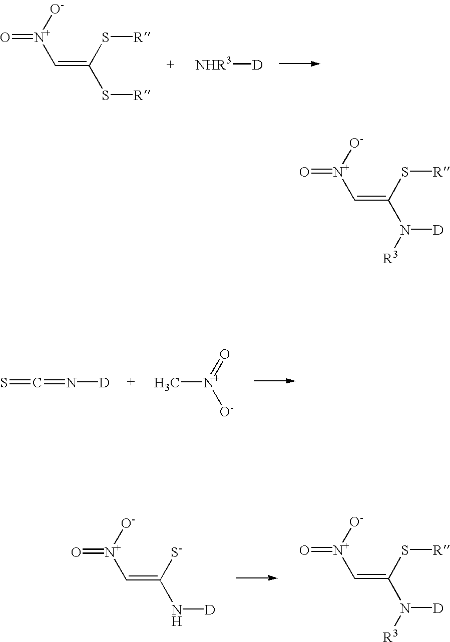

Step B2 is a deprotection (hydrolysis) of the benzoic acid obtained in step B1.

Steps C1 and C2 are as described for the corresponding steps in general procedure (A).

All the steps except steps B1 and B2 are in analogy with similar transformations described in WO 00/69810.

General Procedure (F) for Solution Phase Preparation of Compounds of General Formula (I₂):

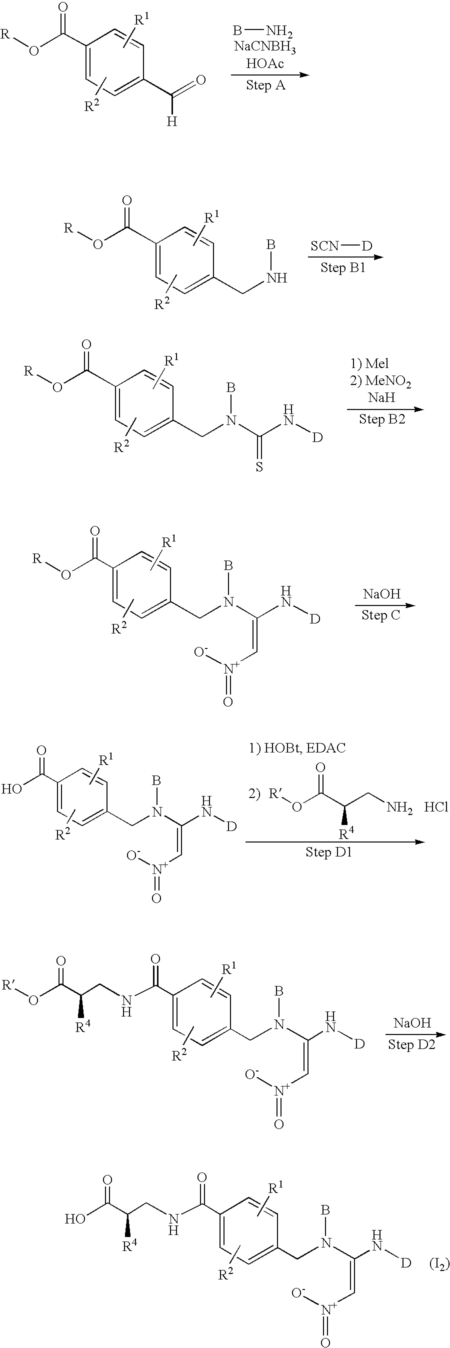

wherein R¹, R², R⁴, B and D are as defined for formula (I), R and R′ are protecting groups such as C₁₋₆-alkyl and may be the same or different.

Step A is as described in step A in general procedure (A).

Step B1 is a thiourea forming reaction of the secondary amine obtained in step A with an isocyanate D-NCS.

In step B2, an alkylation of the thiourea functionality is carried out followed by substitution of the methylthiolate group by the anion of nitromethane as described in Rajappa et al., Indian J. Chem., 1977, Vol. 15B, 297.

Step C is a deprotection (hydrolysis) of the benzoic acid obtained in step B2.

Step D1 is a coupling reaction (amide bond formation) of the benzoic acid intermediate from step C with a 3-aminopropionic acid ester derivative.

Step D2 is a hydrolysis of the 3-aminopropionic acid derivative from step D1 to give the compounds of the general formula (I₂) of the invention.

All steps except steps Bi and B2 are in analogy with similar transformations described in WO 00/69810.

In step C1 of General Procedure (A), (B), (C) and (E) as well as step D1 of General Procedure (D) and (F), the amine component of these steps may be a 5-aminotetrazole resulting in products of the invention wherein A is tetrazolyl.

General Procedure (G) for Solution Phase Preparation of Compounds of General Formula (I₃):

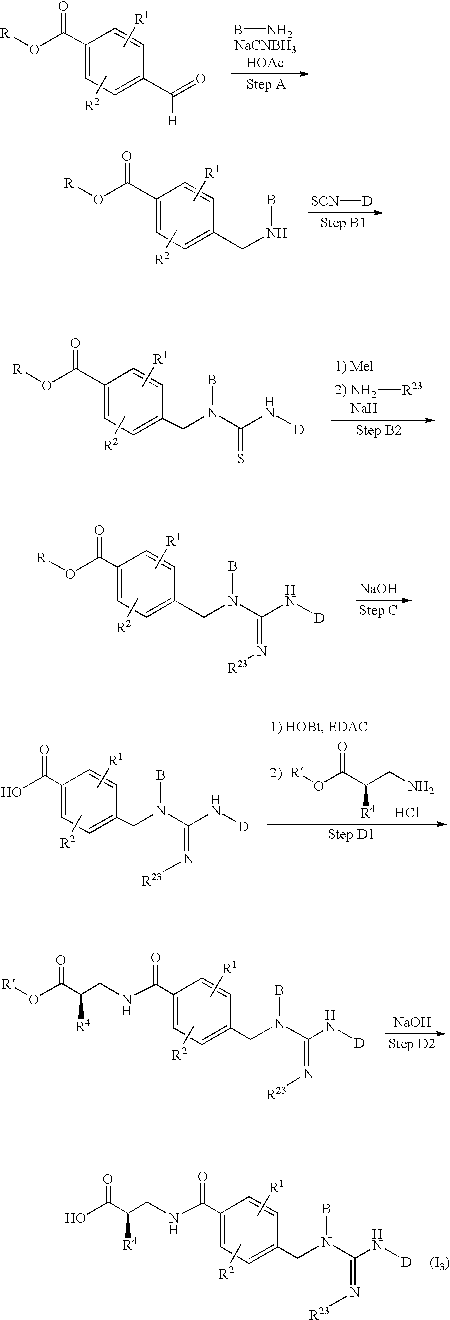

wherein R¹, R², R³, R⁴, B and D are as defined for formula (I), R and R′ are protecting groups such as C₁₋₆-alkyl and may be the same or different. R²³ is cyanomethyl or 2,2,2-trifluoroethylamine.

Step A is as described in step A in general procedure (A).

Step B1 is a thiourea forming reaction of the secondary amine obtained in step A with an isothiocyanate, S═C═N-D.

In step B2, a methylation of the thiourea functionality is carried out followed by substitution of the methylthiolate group by nucleophile R²³—NH₂.

Step C is a deprotection (hydrolysis) of the benzoic acid obtained in step B2.

Step D1 is a coupling reaction (amide bond formation) of the benzoic acid intermediate from step C with a 3-aminopropionic acid ester derivative.

Step D2 is a hydrolysis of the 3-aminopropionic acid derivative from step D1 to give the compounds of the general formula (I₂) of the invention.

All steps except steps B1 and B2 are in analogy with similar transformations described in WO 00/69810.

General Procedure (H) for Solution Phase Preparation of Compounds of General Formula (I₄):

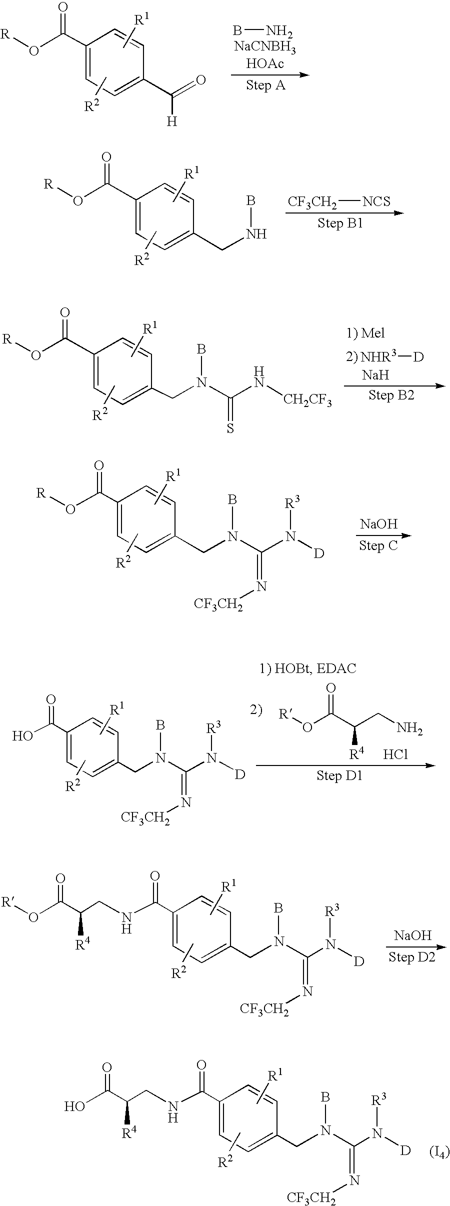

wherein R¹, R², R³, R⁴, B and D are as defined for formula (I), R and R′ are protecting groups such as C₁₋₆-alkyl and may be the same or different.

Step A is as described in step A in general procedure (A).

Step B1 is a thiourea forming reaction of the secondary amine obtained in step A with 2,2,2-trifluoroethyli-thiocyanate as described in S. A. Lee, J. Fluor. Chem., 2001, 55-57.

In step B2, a methylation of the thiourea functionality is carried out followed by substitution of the methylthiolate group by D-NH—R³.

Step C is a deprotection (hydrolysis) of the benzoic acid obtained in step B2.

Step D1 is a coupling reaction (amide bond formation) of the benzoic acid intermediate from step C with a 3-aminopropionic acid ester derivative.

Step D2 is a hydrolysis of the 3-aminopropionic acid ester derivative from step D1 to give the compounds of the general formula (I₂) of the invention.

All steps except steps B1 and B2 are in analogy with similar transformations described in WO 00/69810.

Preferred compounds according to the invention, which may be prepared according to the procedures set forth in this application, includes:

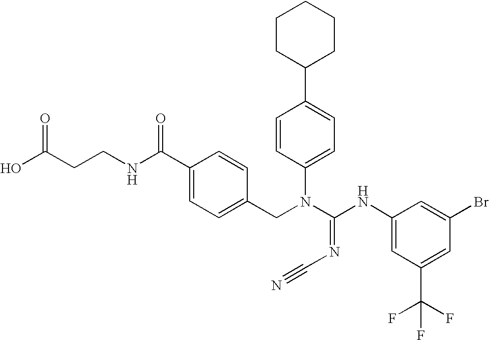

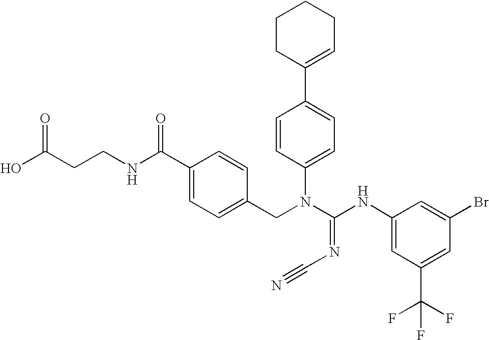

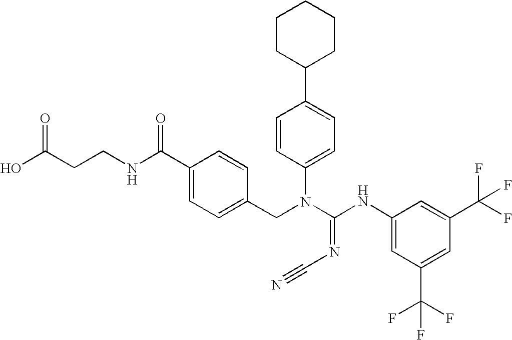

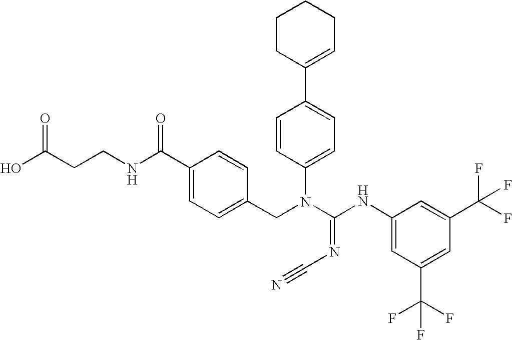

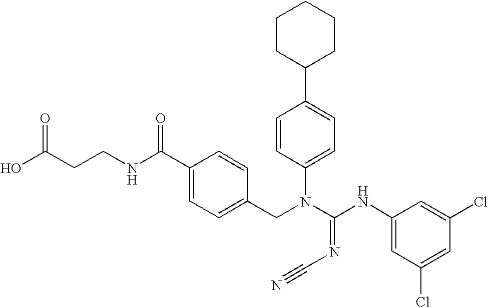

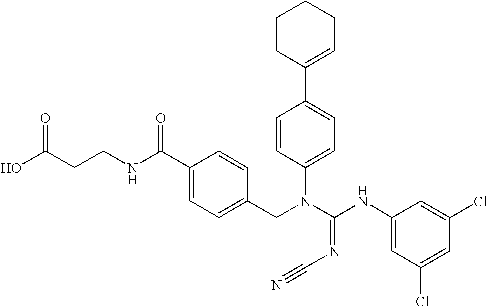

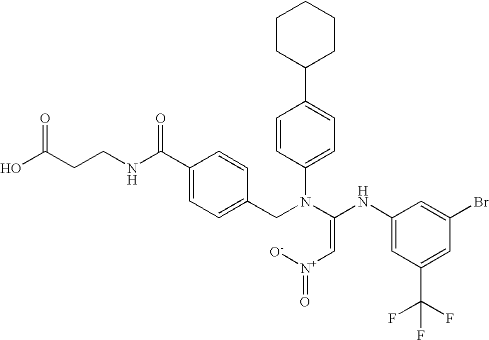

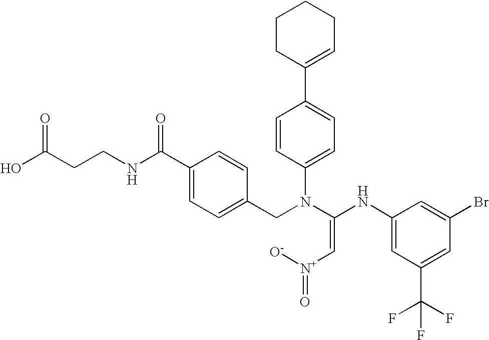

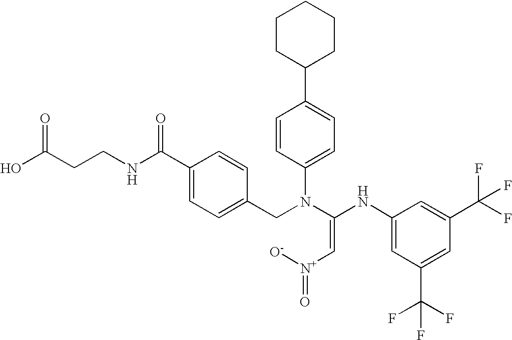

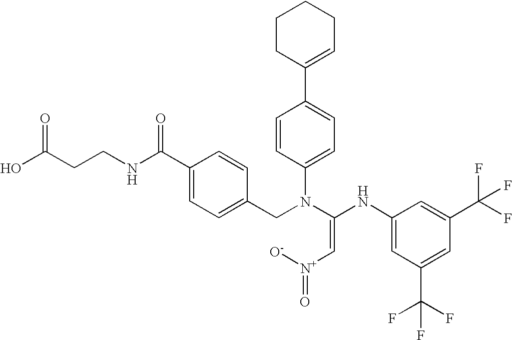

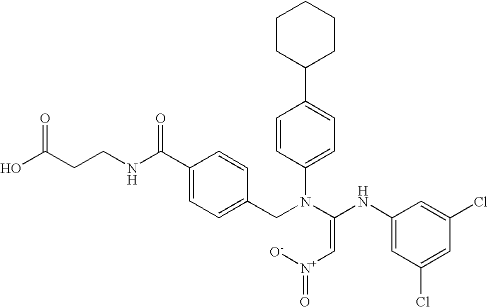

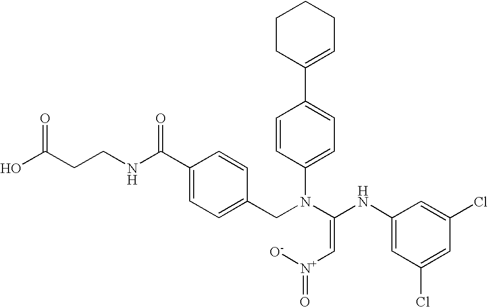

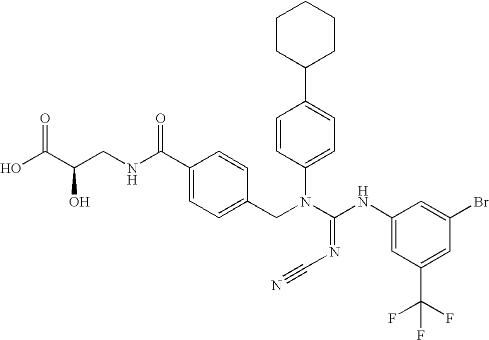

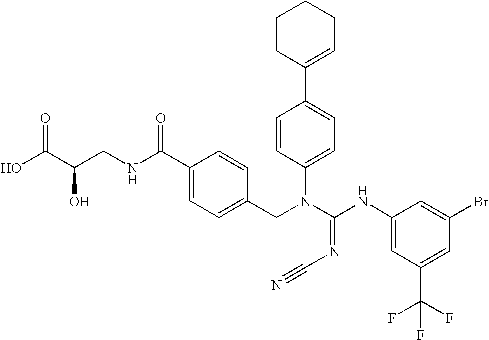

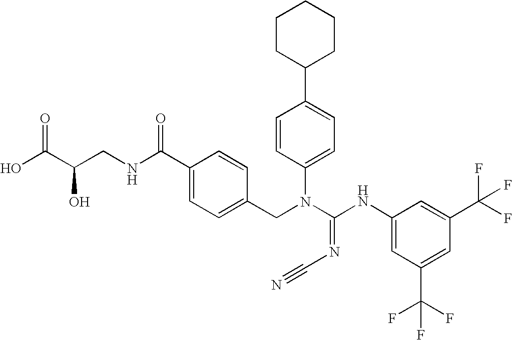

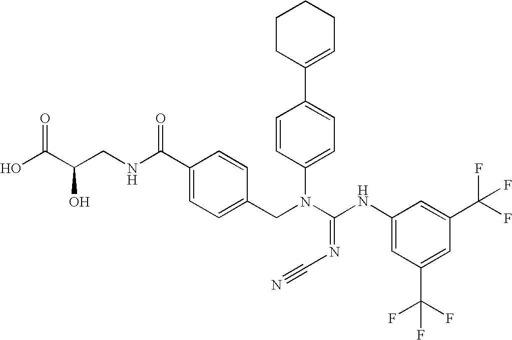

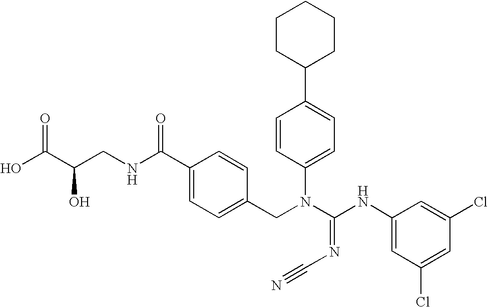

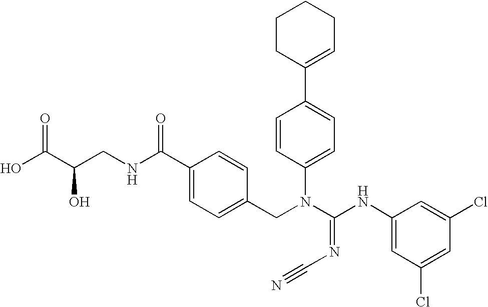

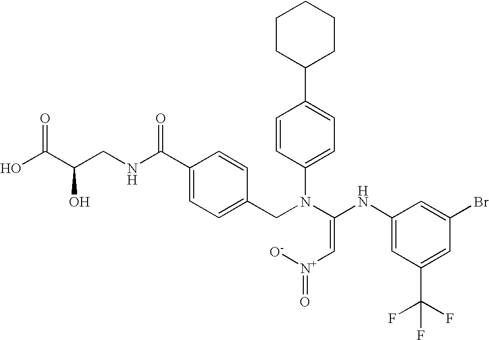

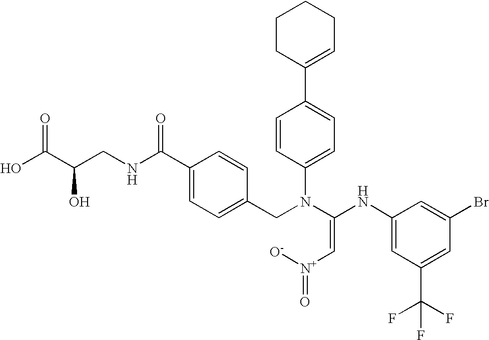

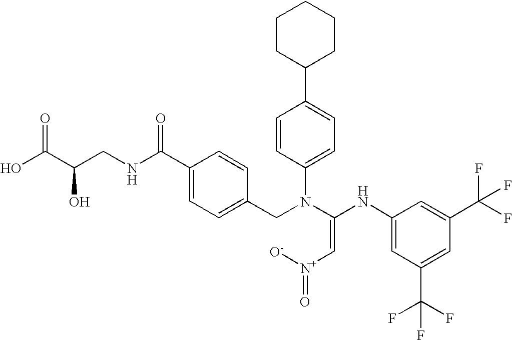

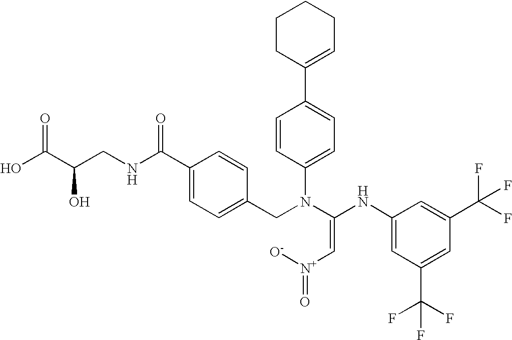

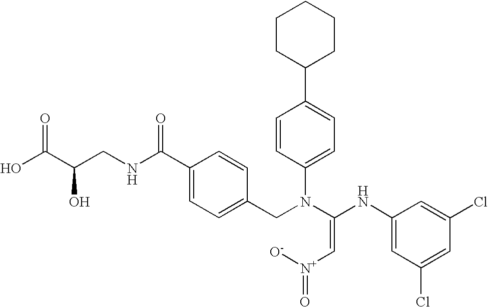

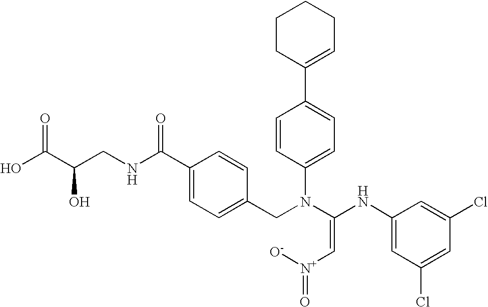

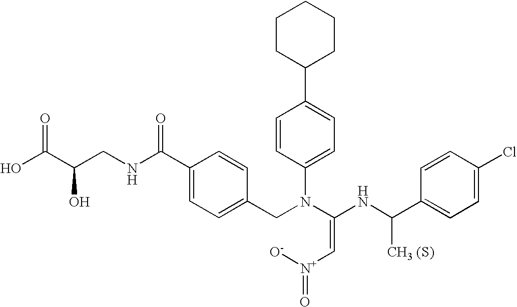

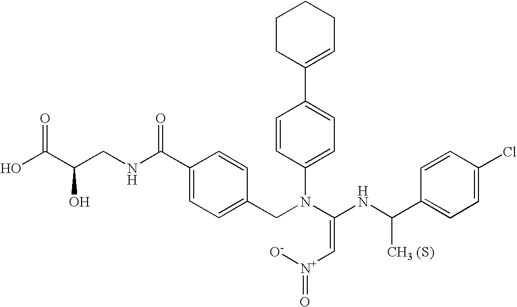

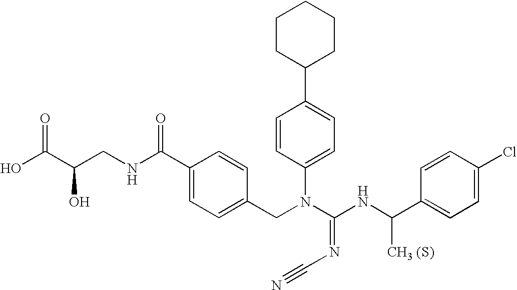

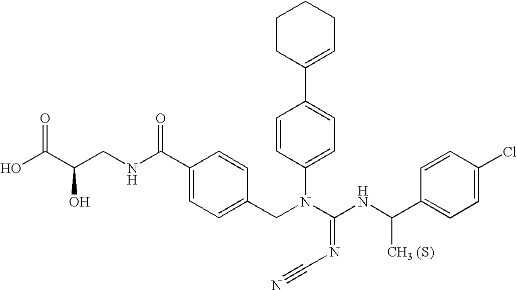

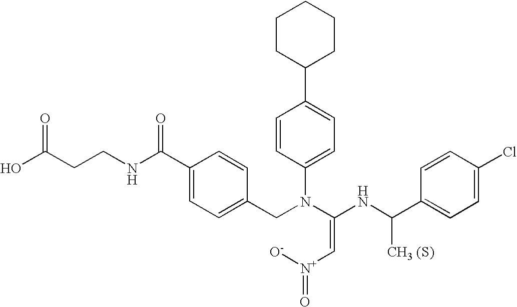

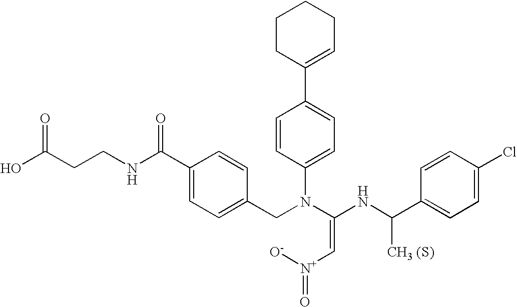

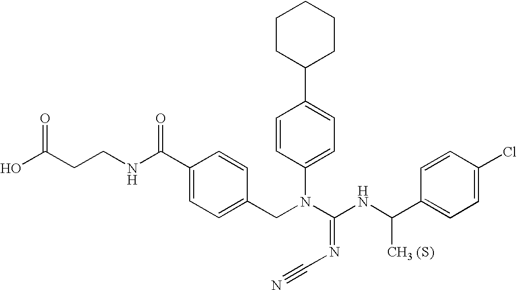

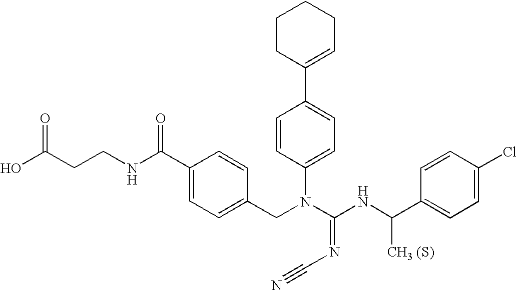

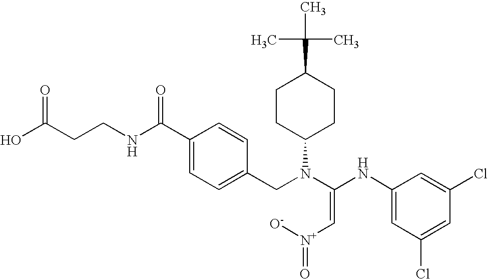

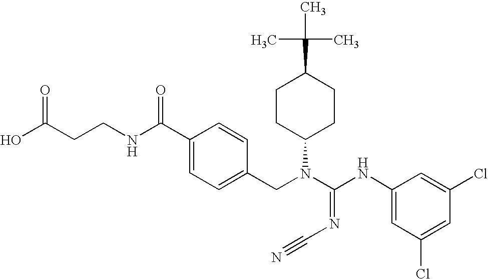

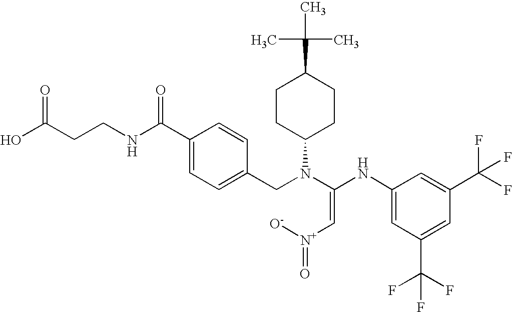

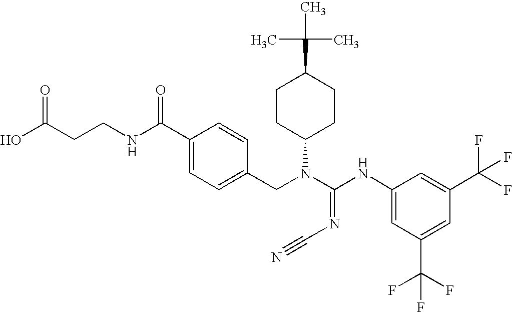

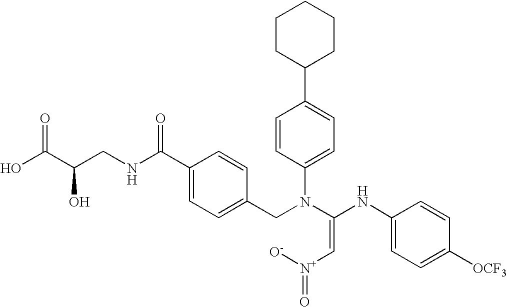

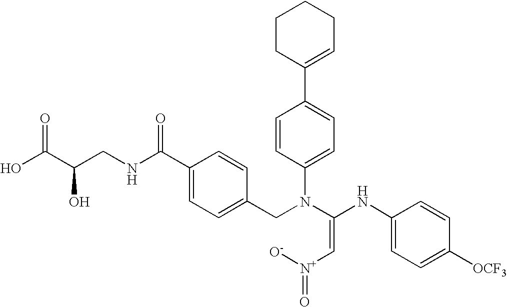

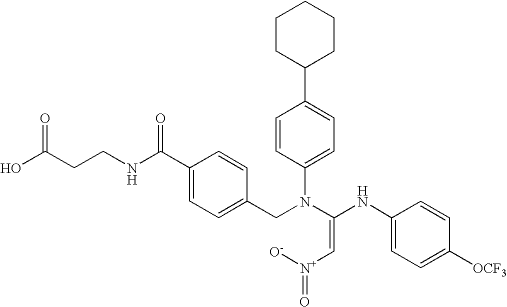

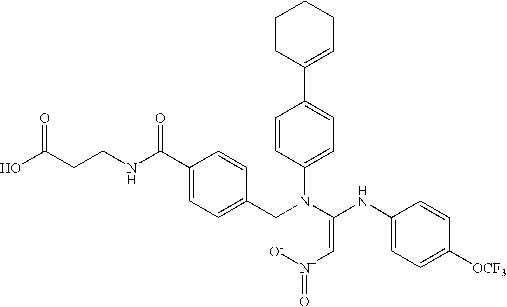

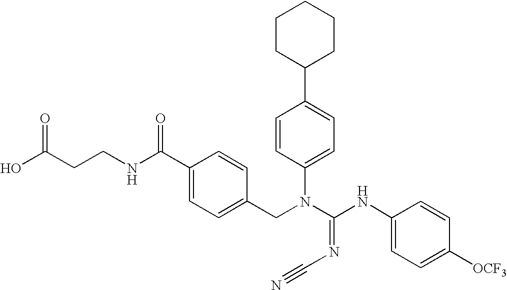

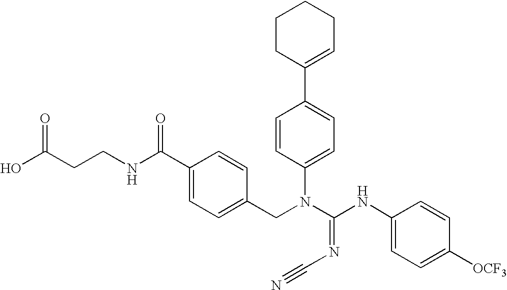

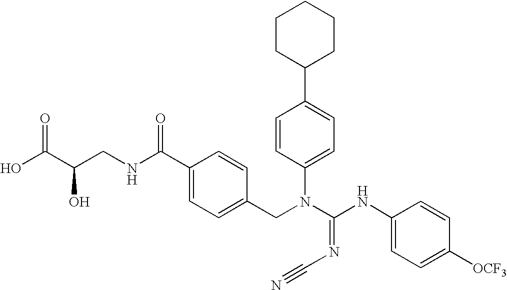

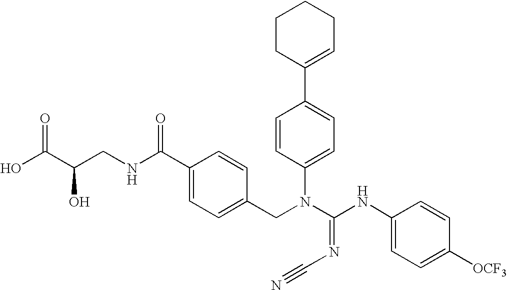

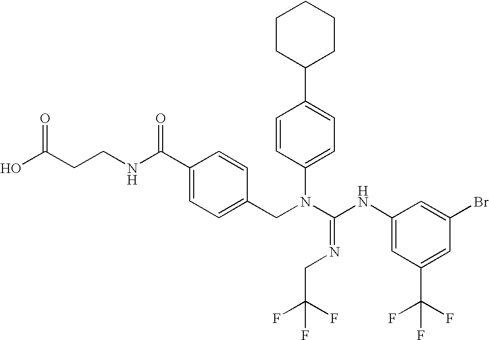

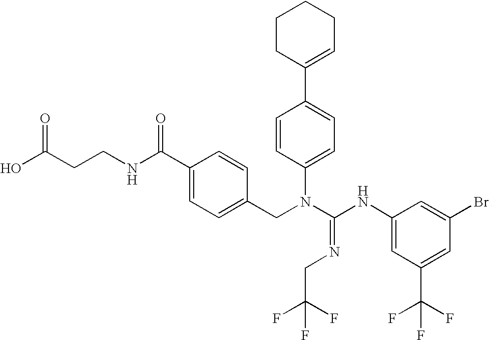

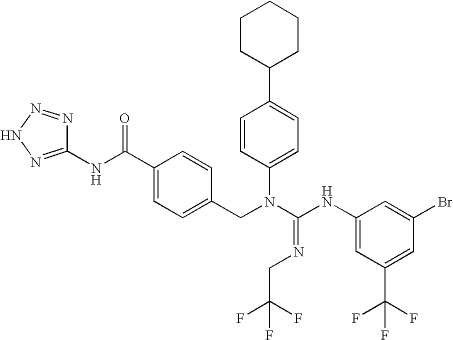

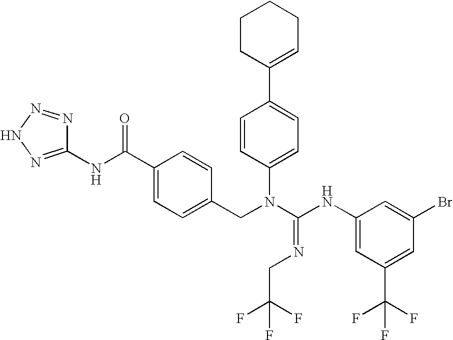

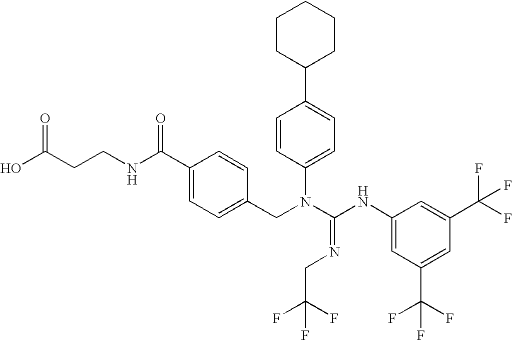

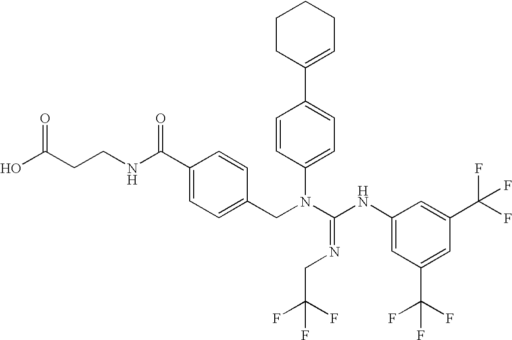

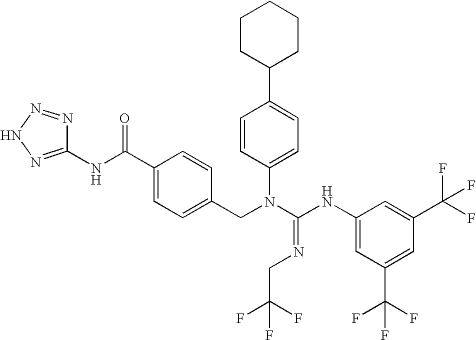

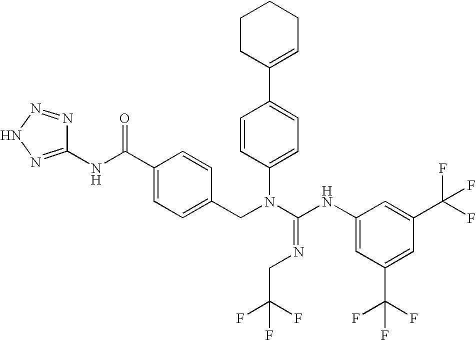

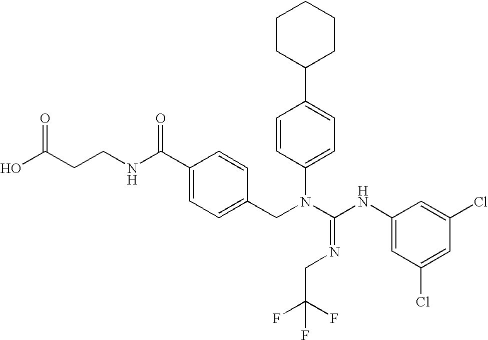

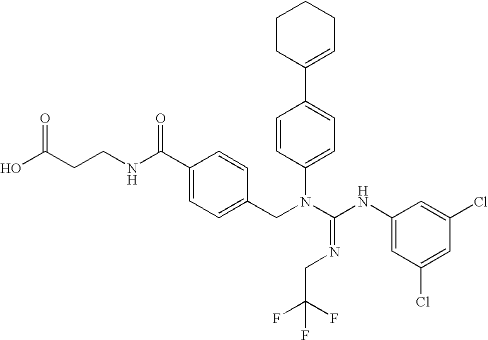

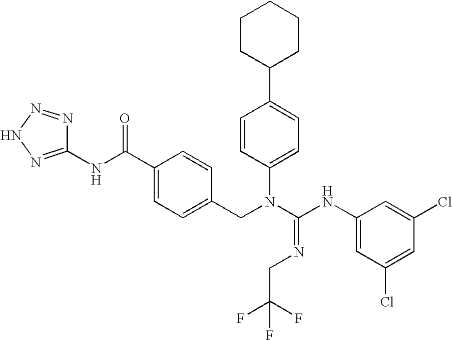

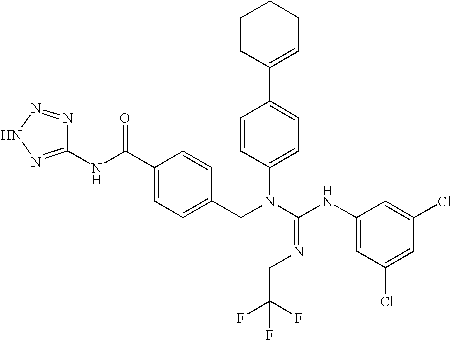

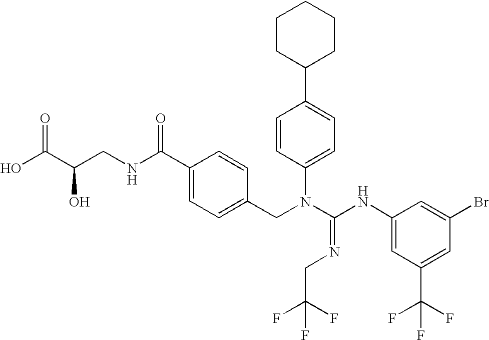

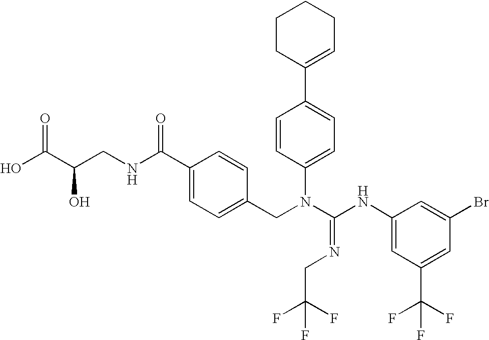

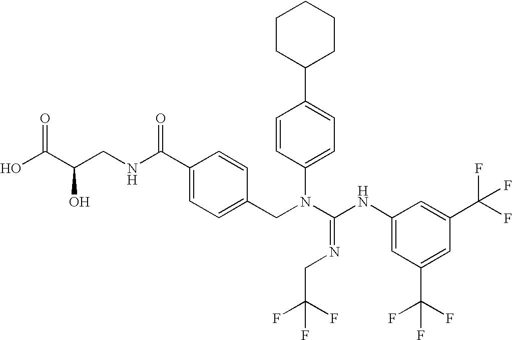

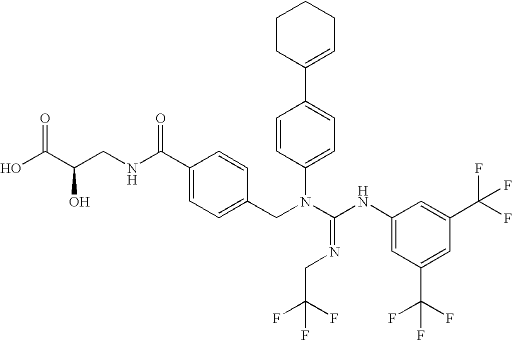

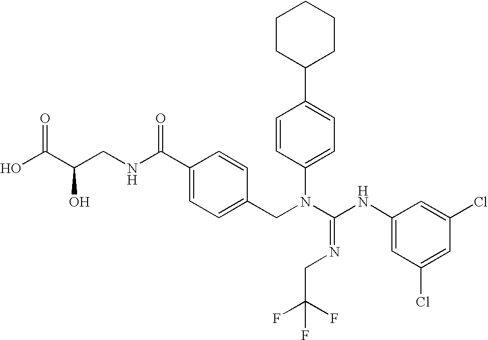

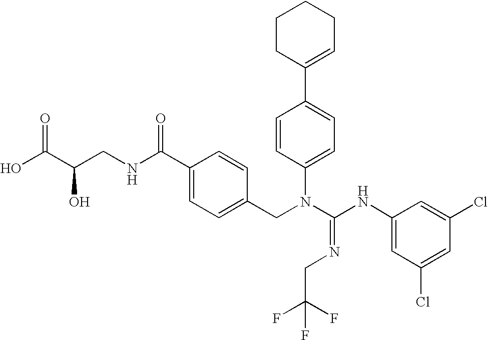

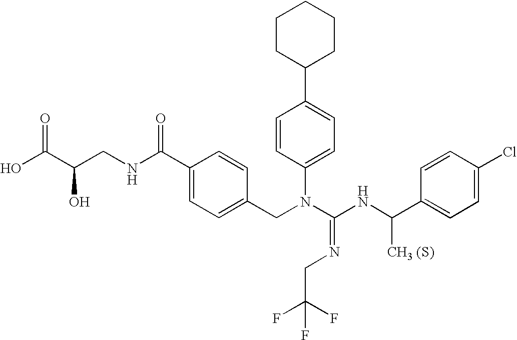

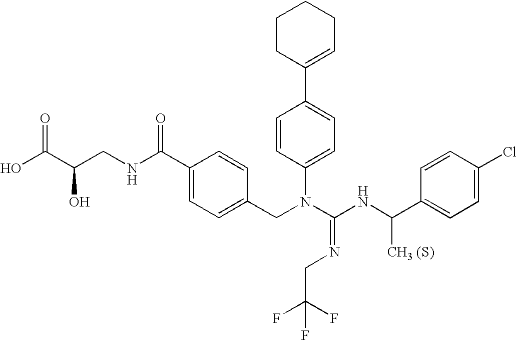

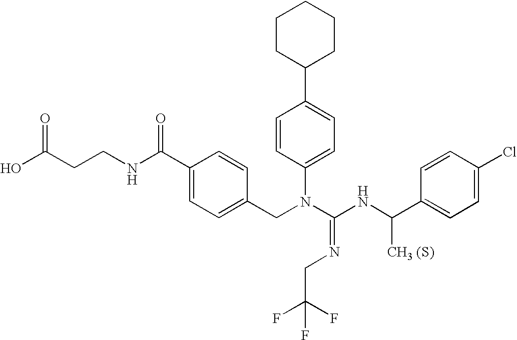

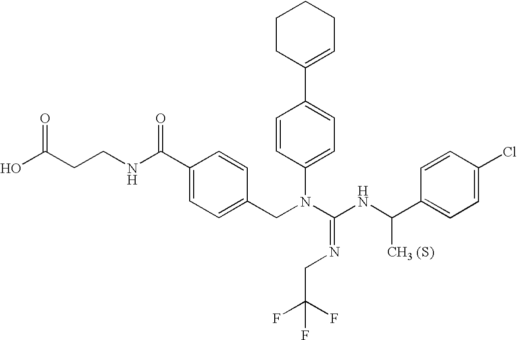

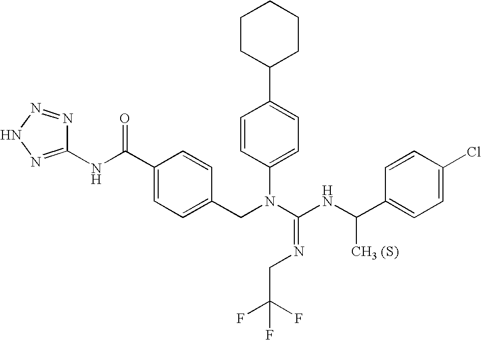

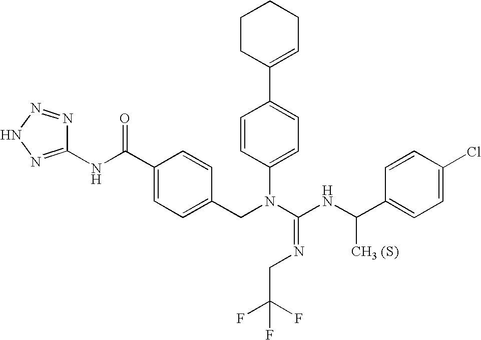

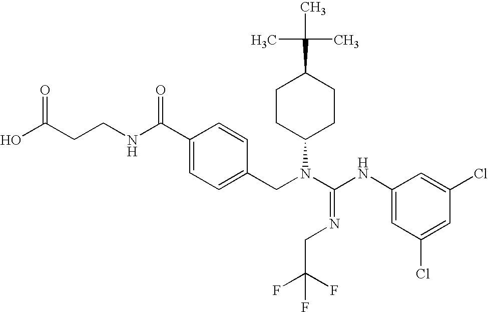

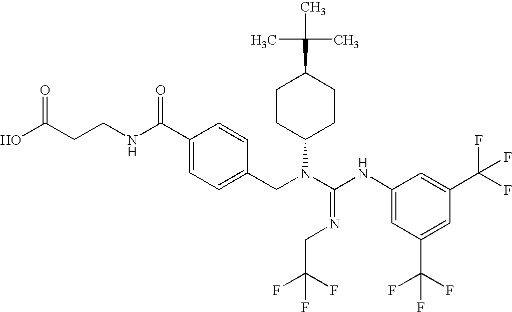

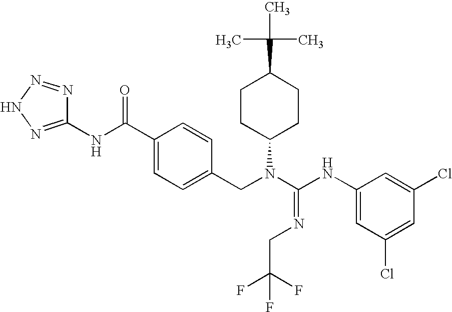

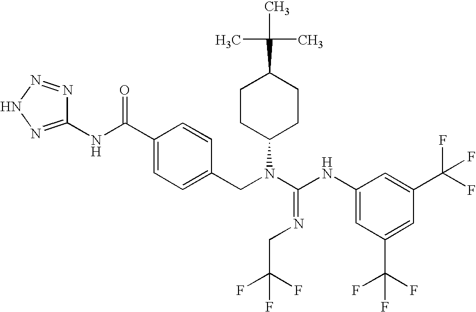

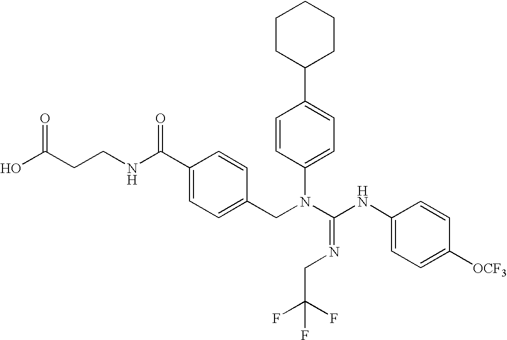

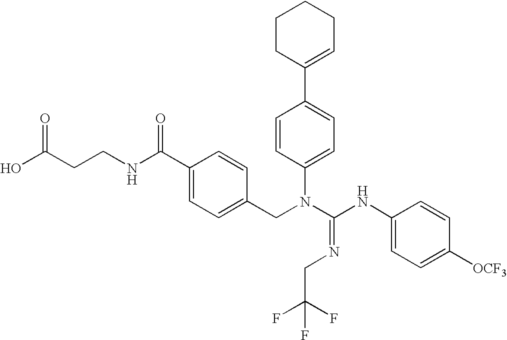

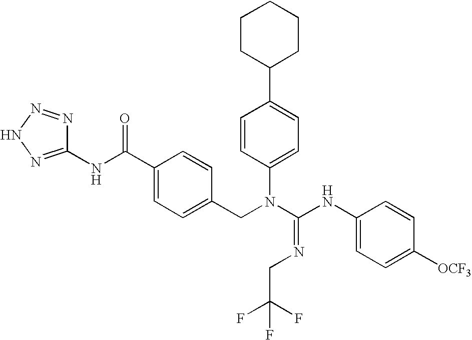

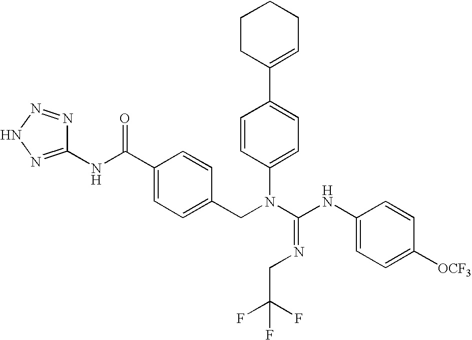

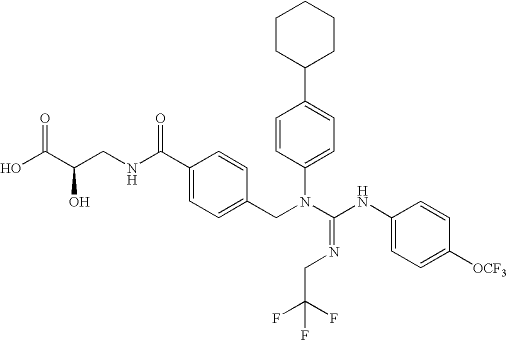

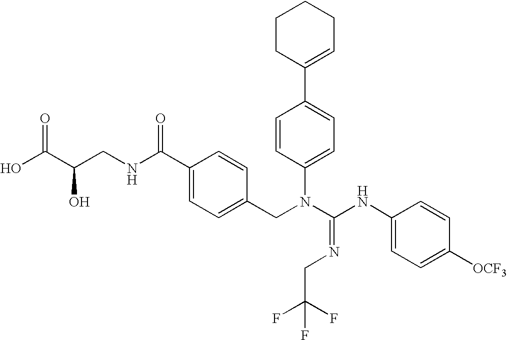

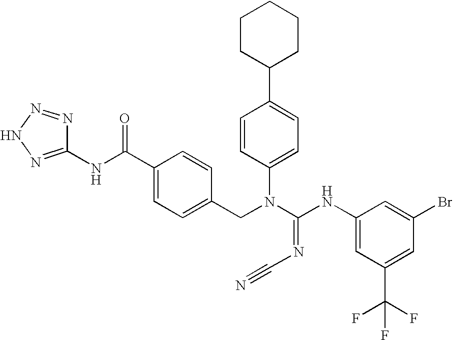

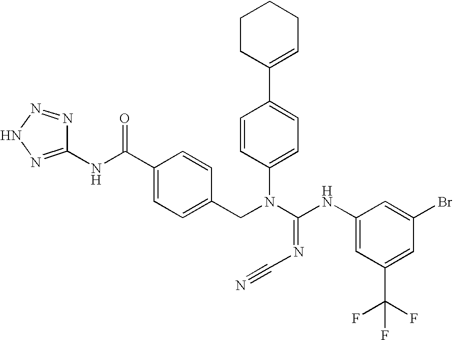

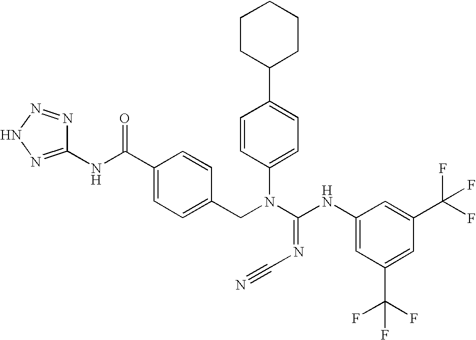

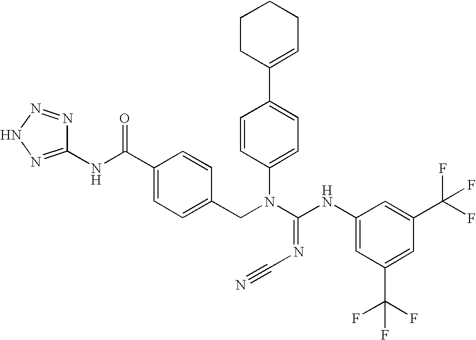

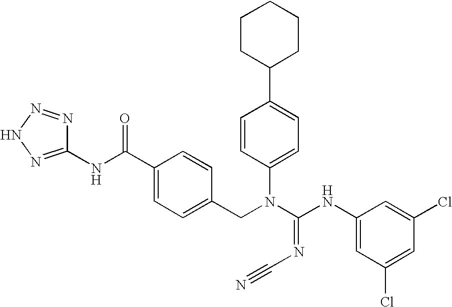

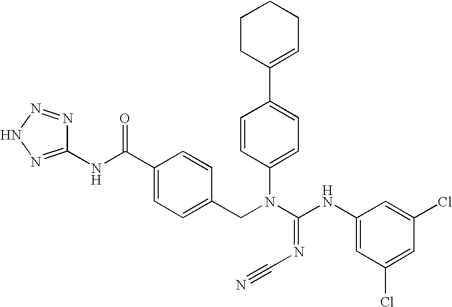

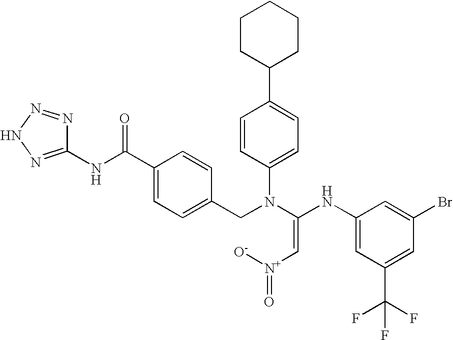

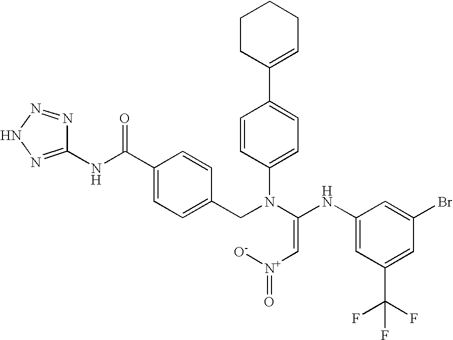

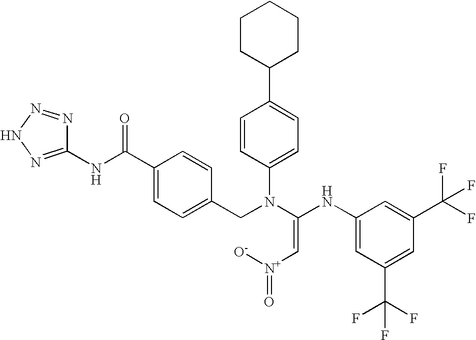

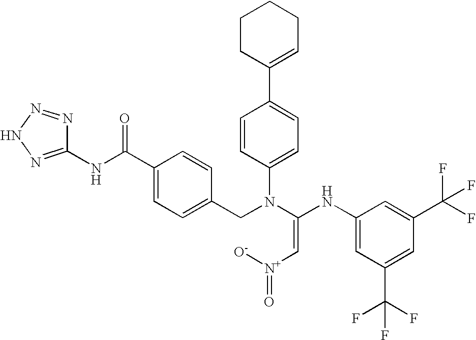

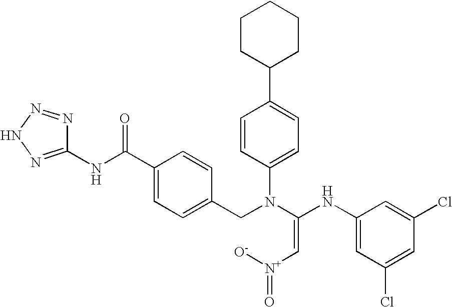

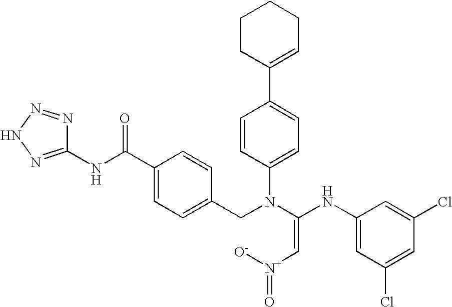

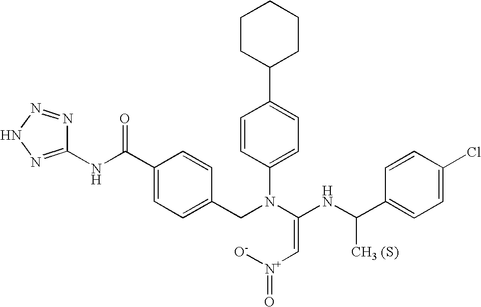

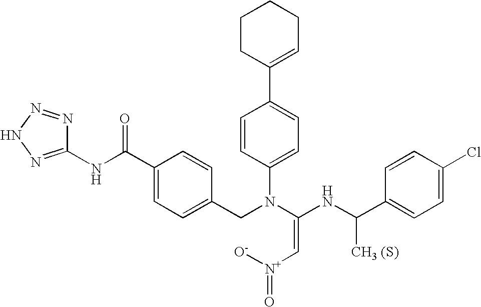

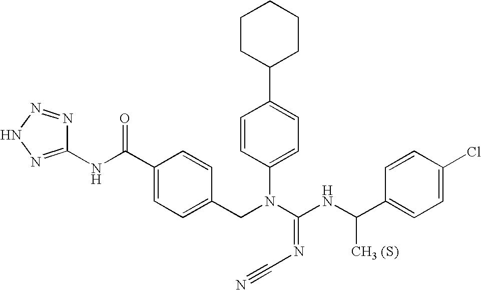

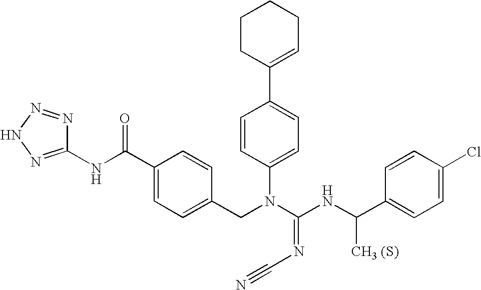

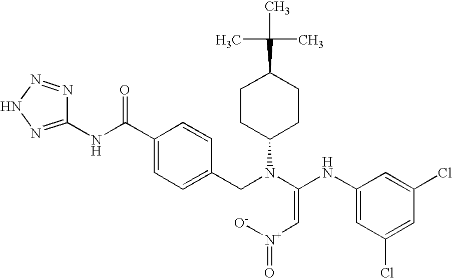

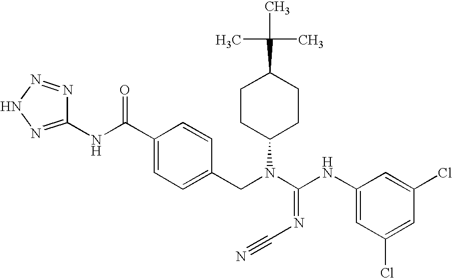

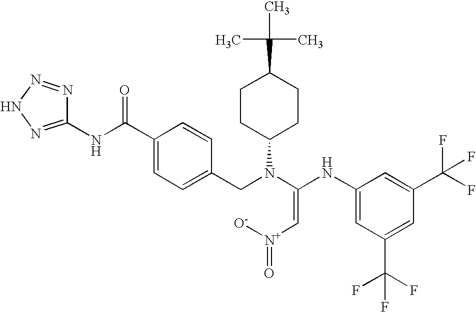

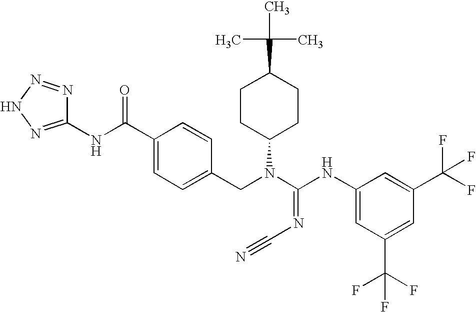

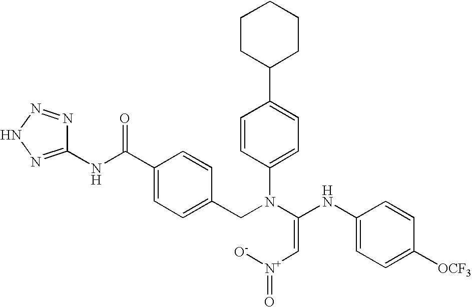

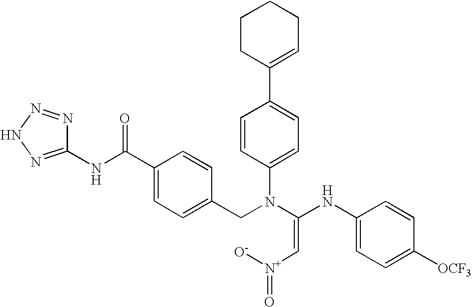

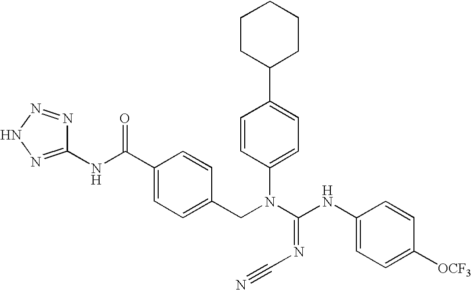

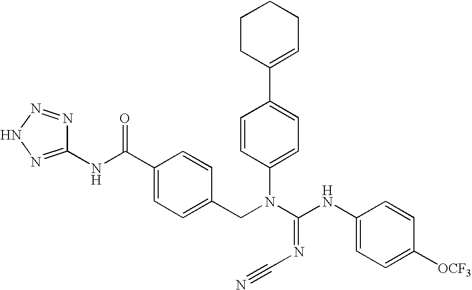

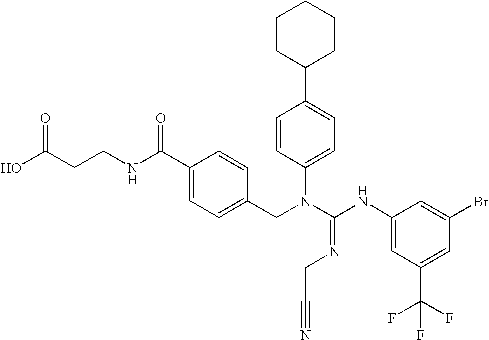

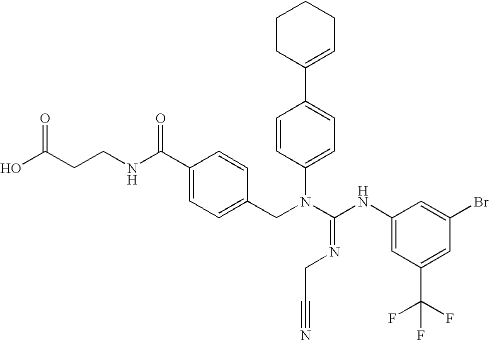

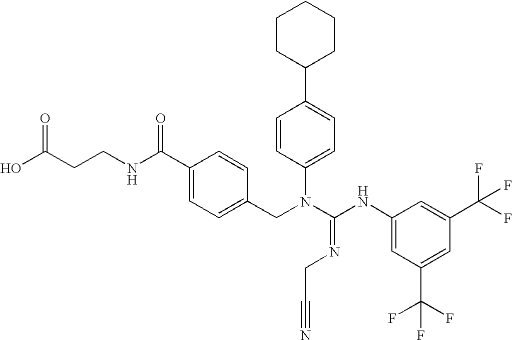

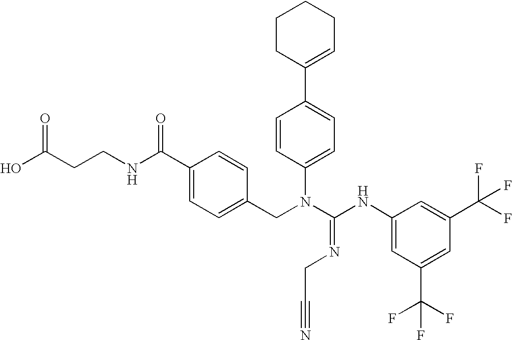

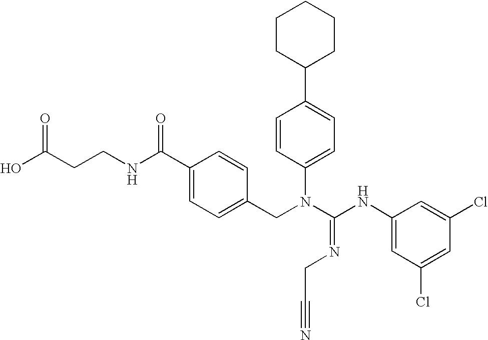

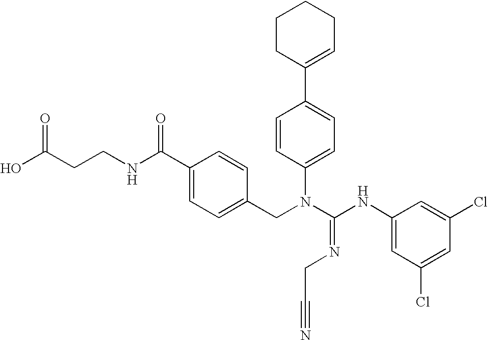

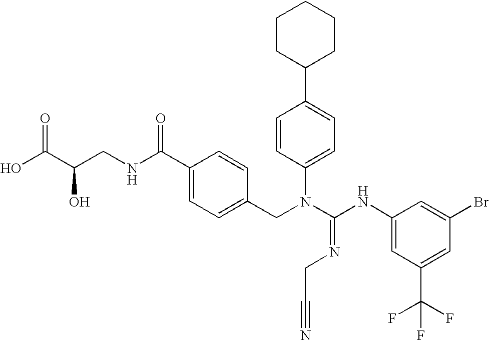

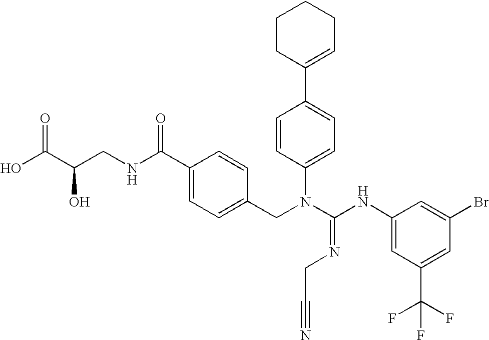

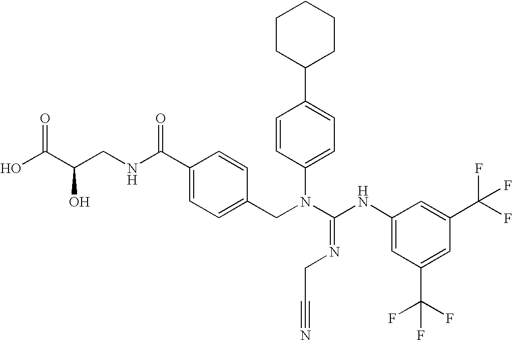

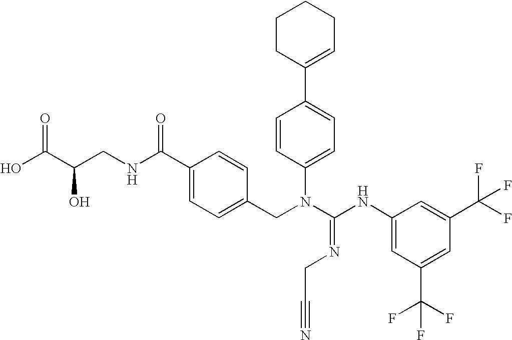

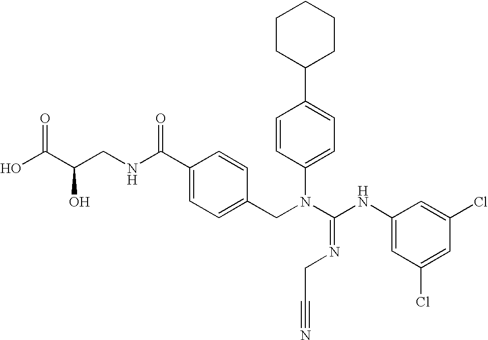

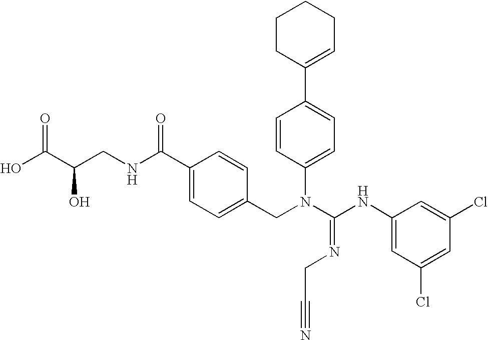

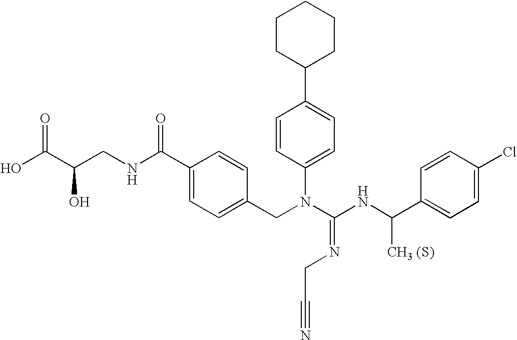

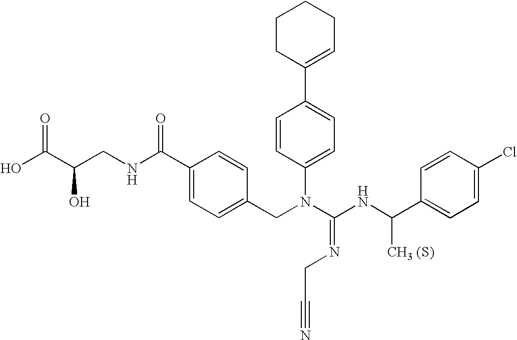

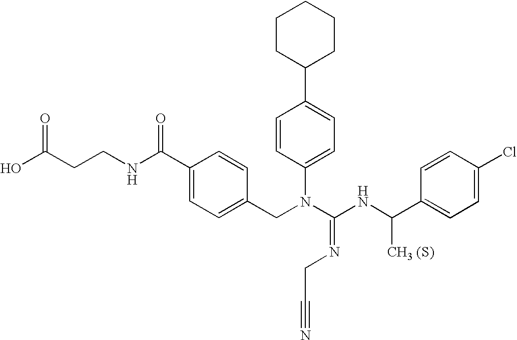

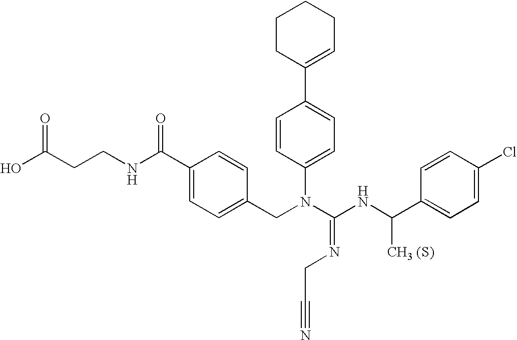

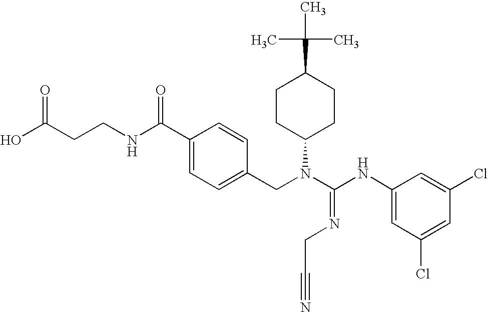

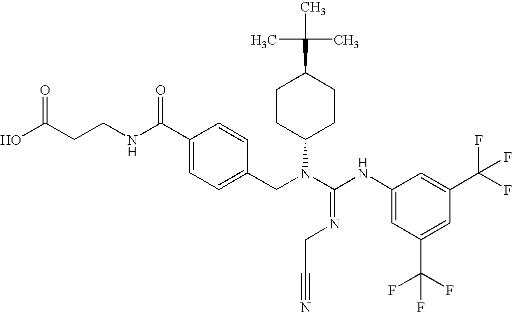

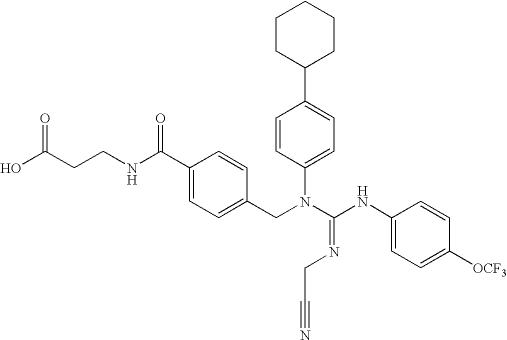

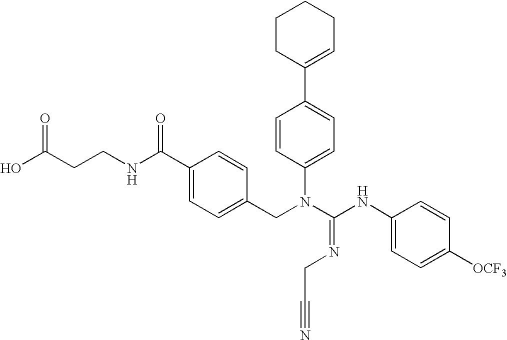

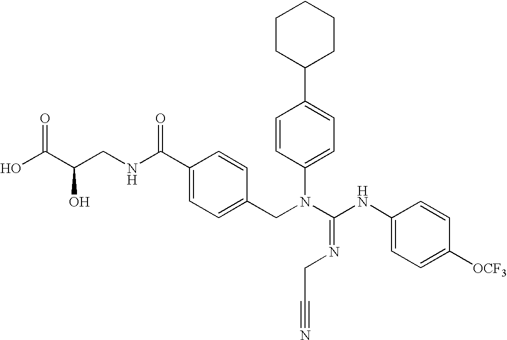

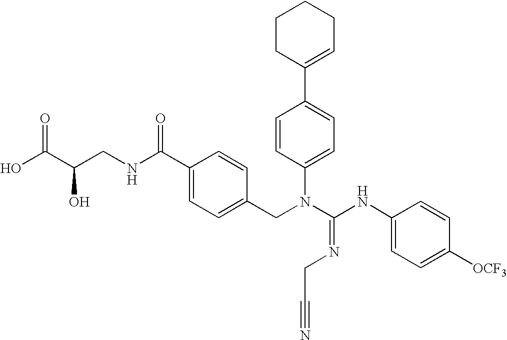

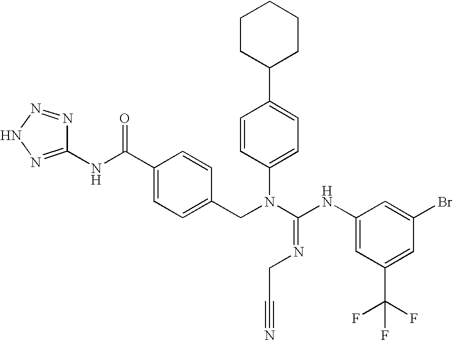

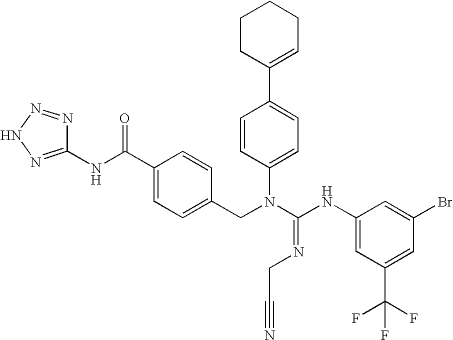

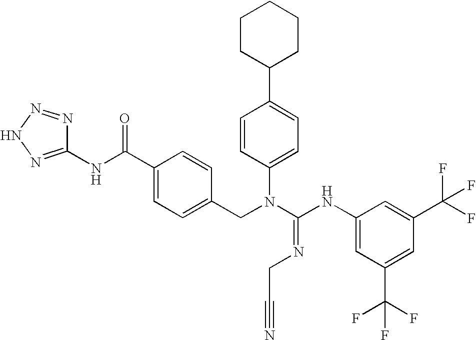

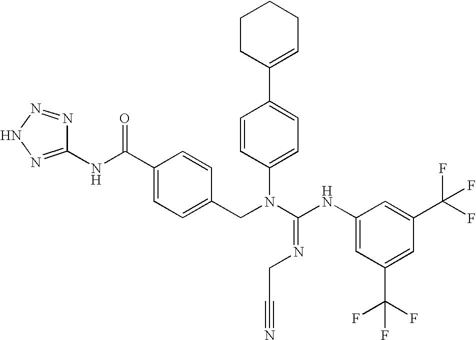

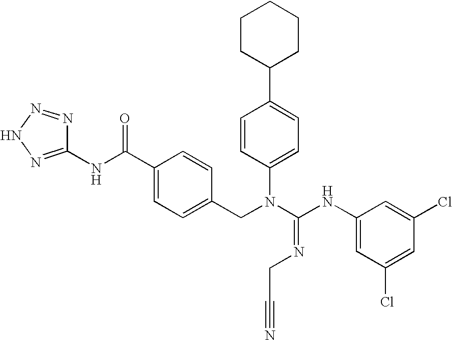

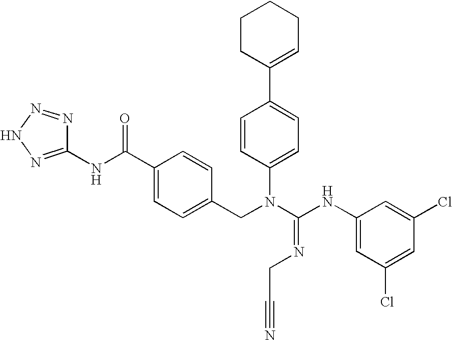

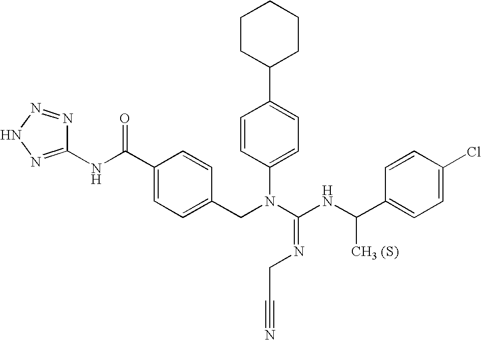

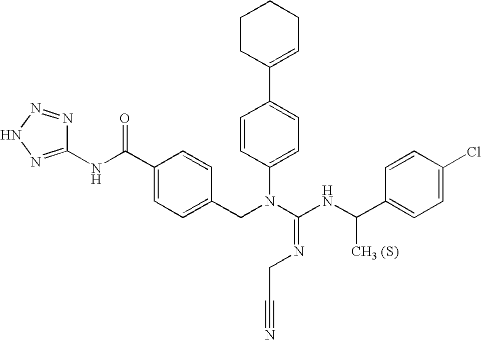

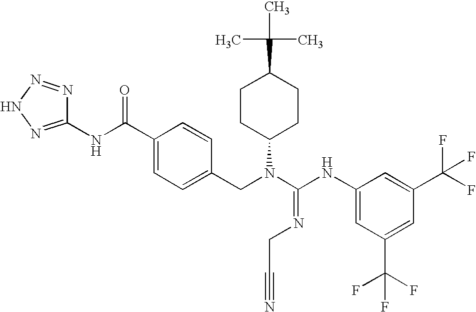

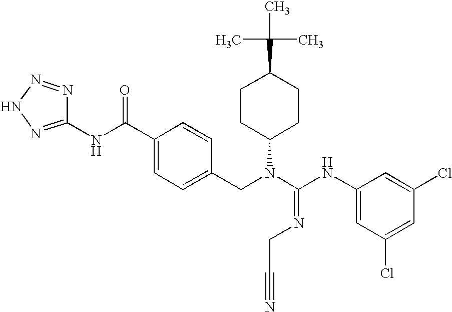

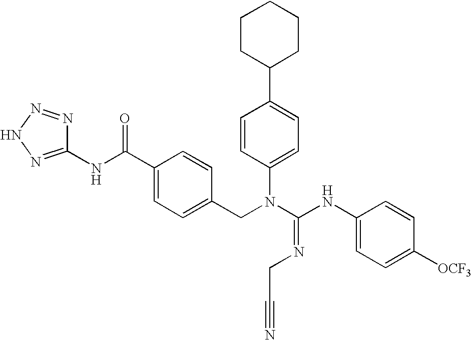

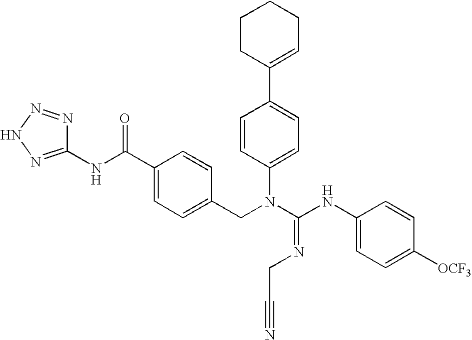

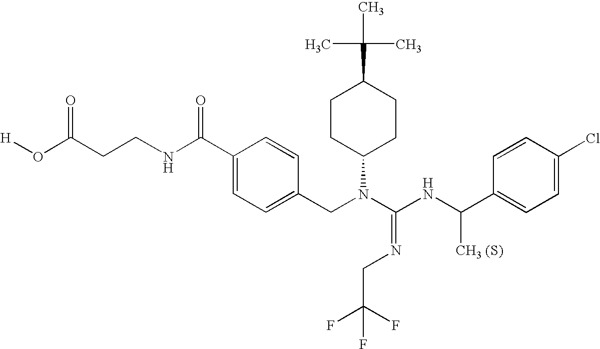

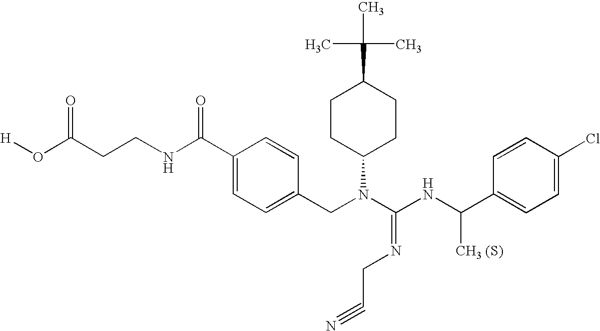

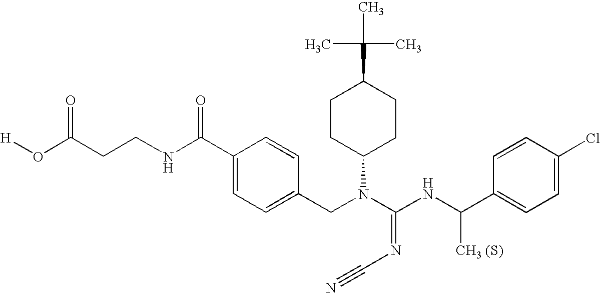

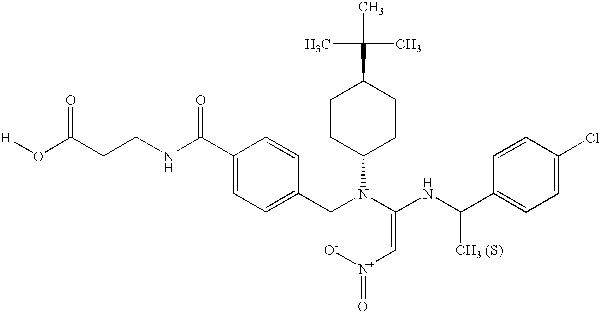

Pharmacological Methods

In the following section binding assays as well as functional assays useful for evaluating the efficiency of the compounds of the invention are described.

Binding of compounds to the glucagon receptor may be determined in a competition binding assay using the cloned human glucagon receptor.

Antagonism may be determined as the ability of the compounds to inhibit the amount of cAMP formed in the presence of 5 nM glucagon.

Glucagon Binding Assay (I)

Receptor binding is assayed using cloned human receptor (Lok et al., Gene 140, 203-209 (1994)). The receptor is inserted in the pLJ6′ expression vector using EcoRI/SSt1 restriction sites (Lok et al.) is expressed in a baby hamster kidney cell line (A3 BHK 570-25). Clones are selected in the presence of 0.5 mg/ml G-418 and are shown to be stable for more than 40 passages. The K_{d} is shown to be 0.1 nM.

Plasma membranes are prepared by growing cells to confluence, detaching them from the surface and resuspending the cells in cold buffer (10 mM tris/HCl, pH 7.4 containing 30 mM NaCl, 1 mM dithiothreitol, 5 mg/l leupeptin (Sigma), 5 mg/l pepstatin (Sigma), 100 mg/l bacitracin (Sigma) and 15 mg/l recombinant aprotinin (Novo Nordisk A/S)), homogenization by two 10-s bursts using a Polytron PT 10-35 homogenizer (Kinematica), and centrifugation upon a layer of 41 w/v % sucrose at 95.000× g for 75 min. The white band located between the two layers is diluted in buffer and centrifuged at 40.000× g for 45 min. The precipitate containing the plasma membranes is suspended in buffer and stored at −80° C. until use.

Glucagon is iodinated according to the chloramine T method (Hunter and Greenwood, Nature 194, 495 (1962)) and purified using anion exchange chromatography (Jørgensen et al., Hormone and Metab. Res. 4, 223-224 (1972). The specific activity is 460 μCi/μg on the day of iodination. Tracer is stored at −18° C. in aliquots and are used immediately after thawing.

Binding assays are carried out in triplicate in filter microtiter plates (MADV N65, Millipore). The buffer used in this assay is 50 mM HEPES, 5 mM EGTA, 5 mM MgCl₂, 0.005% tween 20, pH 7.4. Glucagon is dissolved in 0.05 M HCl, added an equal amount (w/w) of human serum albumin and freeze-dried. On the day of use, it is dissolved in water and diluted in buffer to the desired concentrations.

Test compounds are dissolved and diluted in DMSO. 140 μl buffer, 25 μl glucagon or buffer, and 10 μl DMSO or test compound are added to each well. Tracer (50.000 cpm) is diluted in buffer and 25 μl are added to each well. 1-4 μg freshly thawed plasma membrane protein diluted in buffer is then added in aliquots of 25 μl to each well. Plates are incubated at 30° C. for 2 hours. Non-specific binding is determined with 10⁻⁶ M of glucagon. Bound tracer and unbound tracer are then separated by vacuum filtration (Millipore vacuum manifold). The plates are washed with 2×100 μl buffer/well. The plates are air dried for a couple of hours, whereupon the filters are separated from the plates using a Millipore Puncher. The filters are counted in a gamma counter.

Functional Assay (I)

The functional assay is carried out in 96 well microtiter plates (tissue culture plates, Nunc). The resulting buffer concentrations in the assay are 50 mM tris/HCl, 1 mM EGTA, 1.5 mM MgSO₄, 1.7 mM ATP, 20 μM GTP, 2 mM IBMX, 0.02% tween-20 and 0.1% human serum albumin. pH is 7.4. Glucagon and proposed antagonist are added in aliquots of 35 μl diluted in 50 mM tris/HCl, 1 mM EGTA, 1.85 mM MgSO₄, 0.0222% tween-20 and 0.111% human serum albumin, pH 7.4. 20 μl of 50 mM tris/HCl, 1 mM EGTA, 1.5 mM MgSO₄, 11.8 mM ATP, 0.14 mM GTP, 14 mM IBMX and 0.1% human serum albumin, pH 7.4 is added. GTP is dissolved immediately before the assay.

50 μl containing 5 μg of plasma membrane protein is added in a tris/HCl, EGTA, MgSO₄, human serum albumin buffer (the actual concentrations are dependent upon the concentration of protein in the stored plasma membranes).

The total assay volume is 140 μl. The plates are incubated for 2 hours at 37° C. with continuous shaking. Reaction is terminated by addition of 25 μl 0.5 N HCl. cAMP is measured by the use of a scintillation proximity kit (Amersham).

Glucagon Binding Assay (II)

BHK (baby hamster kidney cell line) cells are transfected with the human glucagon receptor and a membrane preparation of the cells is prepared. Wheat Germ Agglutinin derivatized SPA beads containing a scintillant (WGA beads) (Amersham) bound the membranes. ¹²⁵I-glucagon bound to human glucagon receptor in the membranes and excited the scintillant in the WGA beads to light emission. Glucagon or samples binding to the receptor competed with ¹²⁵I-glucagon.

All steps in the membrane preparation are kept on ice or performed at 4° C. BHK cells are harvested and centrifuged. The pellet is resuspended in homogenisation buffer (25 mM HEPES, pH=7.4, 2.5 mM CaCl₂, 1.0 mM MgCl₂, 250 mg/l bacitracin, 0,1 mM Pefabloc), homogenised 2×10 sec using Polytron 10-35 homogenizer (Kinematica) and added the same amount of homogenisation buffer as used for resuspension. After centrifugation (15 min at 2000× g) the supernatant is transferred to cold centrifuge tubes and centrifuged for 45 min at 40.000× g. The pellet is resuspended in homogenisation buffer, homogenised 2×10 sec (Polytron) and additional homogenisation buffer is added. The suspension is centrifuged for 45 min at 40.000× g and the pellet is resuspended in resuspension buffer (25 mM HEPES, pH=7.4, 2.5 mM CaCl₂, 1.0 mM MgCl₂) and homogenised 2×10 sec. (Polytron). The protein concentration is normally around 1.75 mg/ml. Stabilisation buffer (25 mM HEPES, pH=7.4, 2.5 mM CaCl₂, 1.0 mM MgCl₂, 1% bovine serum albumin, 500 mg/l bacitracin, 2.5 M sucrose) is added and the membrane preparation is stored at −80° C.

The glucagon binding assay is carried out in opti plates (Polystyrene Microplates, Packard). 50 μl assay buffer (25 mM HEPES, pH=7.5, 2.5 mM CaCl₂, 1.0 mM MgCl₂, 0.003% Tween-20, 0.005% bacitracin, 0.05% sodium azide) and 5 μl glucagon or test compound (in DMSO) are added to each well. 50 μl tracer (¹²⁵1-porcine glucagon, 50.000 cpm) and 50 μl membranes (7.5 μg) containing the human glucagon receptor are then added to the wells. Finally 50 μl WGA beads containing 1 mg beads are transferred to the well. The opti plates are incubated for 4 hours on a shaker and then settled for 8-48 hours. The opti plates are counted in a Topcounter. Non-specific binding is determined with 500 nM of glucagon.

GIP Binding Assay

BHK (baby hamster kidney cell line) cells are transfected with the human GIP receptor and a membrane preparation of the cells is prepared. Wheat Germ Agglutinin derivatized SPA beads containing a scintillant (WGA beads) (Amersham) bound the membranes. ¹²⁵I-GIP bound to human GIP receptor in the membranes and excited the scintillant in the WGA beads to light emission. GIP or samples binding to the receptor competed with ¹²⁵I-GIP.

All steps in the membrane preparation are kept on ice or performed at 4° C. BHK cells are harvested and centrifuged. The pellet is resuspended in homogenisation buffer (25 mM HEPES, pH=7.4, 2.5 mM CaCl₂, 1.0 mM MgCl₂, 250 mg/l bacitracin, 0.1 mM Pefabloc), homogenised 2×10 sec using Polytron 10-35 homogenizer (Kinematica) and added the same amount of homogenisation buffer as used for resuspension. After centrifugation (15 min at 2000× g) the supernatant is transferred to cold centrifuge tubes and centrifuged for 45 min at 40.000× g. The pellet is resuspended in homogenisation buffer, homogenised 2×10 sec (Polytron) and additional homogenisation buffer is added. The suspension is centrifuged for 45 min at 40.000× g and the pellet is resuspended in resuspension buffer (25 mM HEPES, pH=7.4, 2.5 mM CaCl₂, 1.0 mM MgCl₂) and homogenised 2×10 sec. (Polytron). The protein concentration is normally around 1.75 mg/ml. Stabilisation buffer (25 mM HEPES, pH=7.4, 2.5 mM CaCl₂, 1.0 mM MgCl₂, 1% bovine serum albumin, 500 mg/l bacitracin, 2.5 M sucrose) is added and the membrane preparation is stored at −80° C.

The GIP binding assay is carried out in opti plates (Polystyrene Microplates, Packard). 50 μl assay buffer (25 mM HEPES, pH=7.5, 2.5 mM CaCl₂, 1.0 mM MgCl₂, 0.003% Tween-20, 0.005% bacitracin, 0.05% sodium azide) and 5 μl GIP or test compound (in DMSO) are added to each well. 50 μl tracer (¹²⁵I-porcine GIP, 50.000 cpm) and 50 μl membranes (20 μg) containing the human GIP receptor are then added to the wells. Finally 50 μl WGA beads containing 1 mg beads are transferred to the well. The opti plates are incubated for 3.5 hours on a shaker and then settled for 8-48 hours. The opti plates are counted in a Topcounter. Non-specific binding is determined with 500 nM of GIP.

1. A compound of formula (I): 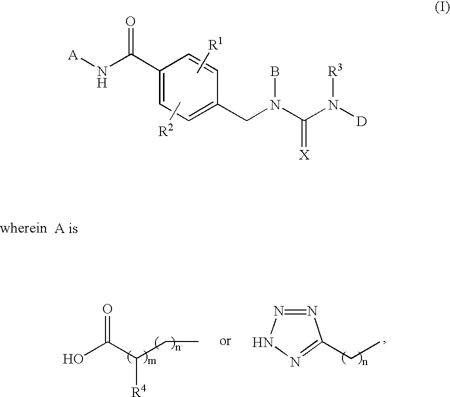
m is 0 or 1,
n is 0, 1, 2 or 3,
with the proviso that m and n must not be 0 at the same time,
R⁴ is hydrogen, fluoro or —(CH₂)ₚ—OR⁵,
p is 0 or 1,
R⁵ is hydrogen, C₁₋₆-alkyl, C₁₋₆-alkanoyl, aryl or aryl-C₁₋₆-alkyl,
R¹ and R² independently are hydrogen, halogen, trifluoromethyl, trifluoromethoxy, cyano, trifluoromethylthio, nitro, C₁₋₆-alkyl, C₁₋₆-alkoxy, hydroxy, C₁₋₆-alkylthio, C₁₋₆-alkylsulfonyl, trifluoromethylsulfonyl or —NR⁶R⁷,
R⁶ and R⁷ independently are hydrogen or C₁₋₆-alkyl,
B is 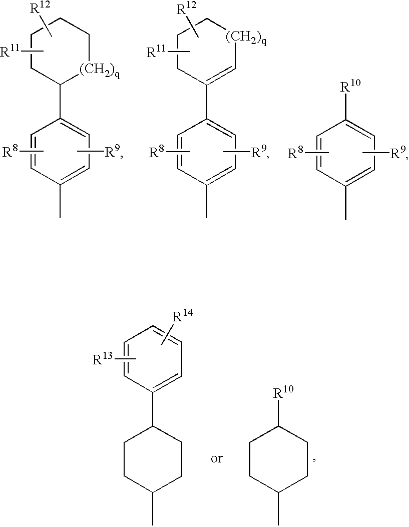
R⁸, R⁹, R¹³ and R¹⁴ independently are hydrogen, halogen, trifluoromethyl, C₁₋₆-alkyl or C₁₋₆-alkoxy,
R¹⁰ is hydrogen, halogen, trifluoromethyl, trifluoromethoxy, cyano, trifluoromethylthio, nitro, C₁₋₆-alkyl, methylthio or C₃₋₈-cycloalkyl,
R¹¹ and R¹² independently are hydrogen or C₁₋₆-alkyl,
q is 0, 1, 2 or 3;
R³ is hydrogen or C₁₋₈-alkyl,
X is ═N—CN, ═N—CH₂R¹⁵, ═CH—NO₂ or ═CHR¹⁵,
R¹⁵ is
hydrogen, cyano or trifluoromethyl,
C₁₋₆-alkyl, which may optionally be substituted with
fluoro, trifluoromethyl, trifluoromethoxy, cyano, trifluoromethylthio, C₁₋₆-alkoxy, hydroxy, C₁₋₆-alkylthio, C₁₋₆-alkylsulfonyl, trifluoromethylsulfonyl or—-NR¹⁶R¹⁷,
R¹⁶ and R¹⁷ independenly are hydrogen or C₁₋₆-alkyl,
aryl or heteroaryl, which may optionally be substituted with
halogen, trifluoromethyl, trifluoromethoxy, cyano, trifluoromethylthio, nitro, C₁₋₆-alkyl, C₁₋₆-alkoxy, hydroxy, C₁₋₆-alkylthio, C₁₋₆-alkylsulfonyl, trifluoromethylsulfonyl or —NR¹⁸R¹⁹,
R¹⁸ and R¹⁹ independenly are hydrogen or C₁₋₆-alkyl,
D is 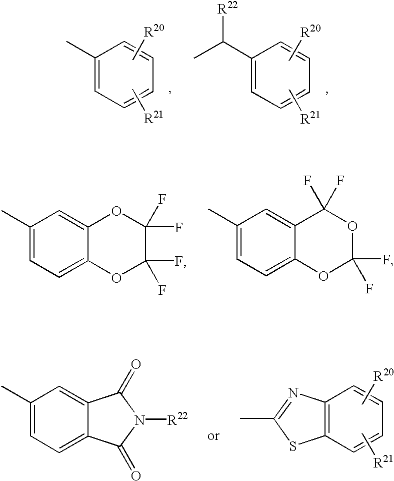
R²⁰ and R²¹ independently are hydrogen, halogen, trifluoromethyl, trifluoromethoxy, cyano, trifluoromethylthio, nitro, C₁₋₆-alkyl, aryl, methylthio, methylsulfonyl, trifluoromethylsulfonyl, —NR²³R²⁴, C₃₋₈-cycloalkyl or —S(O)₂—N(C₁₋₆-alkyl)(aryl),
R²³ and R²⁴ independently are hydrogen or C₁₋₆-alkyl,
R²² is hydrogen, C₁₋₆-alkyl, C₃₋₈-cycloalkyl or C₃₋₈-cycloalkyl-C₁₋₆-alkyl,
as well as any diastereomer or enantiomer or tautomeric form thereof including mixtures of these or a pharmaceutically acceptable salt thereof.

2. A compound according to claim 1, wherein A is

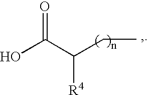

3. A compound according to claim 2, wherein A is

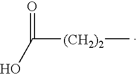

4. A compound according to claim 2, wherein A is

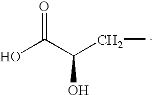

5. A compound according to claim 1, wherein A is

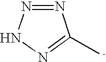

6. A compound according to claim 1, wherein R¹ and R² are both hydrogen.

7. A compound according to claim 1, wherein B is

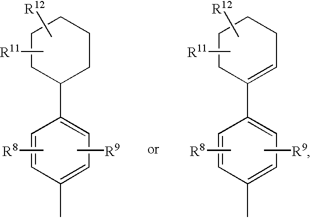

8. A compound according to claim 7, wherein R⁸ and R⁹ are both hydrogen.

9. A compound according to claim 7, wherein R¹¹ and R¹² are both hydrogen.

10. A compound according to claim 1, wherein B is

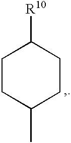

11. A compound according to claim 10, wherein R₁₀ is C₁₋₆-alkyl.

12. A compound according to claim 1, wherein X is ═N—CN, ═CH—NO₂, ═N—CH₂—CF₃ or ═N—CH₂—CN.

13. A compound according to claim 1, wherein R³ is hydrogen.

14. A compound according to claim 1, wherein D is

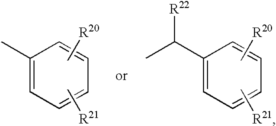

15. A compound according to claim 14, wherein R²⁰ and R²¹ independently are hydrogen, halogen, trifluoromethyl or trifluoromethoxy, and R²² is C₁₋₆-alkyl.

16. A compound according to claim 1, which has an IC₅₀ value of no greater than 5 μM as determined by Glucagon Binding Assay (I) or Glucagon Binding Assay (II).

17. A compound according to claim 16, wherein said compound has an IC₅₀ value of less than 1 μM as determined by Glucagon Binding Assay (I) or Glucagon Binding Assay (II).

18. A compound according to claim 1, which is an agent useful for the treatment of an indication selected from the group consisting of hyperglycemia, impaired glucose tolerance (IGT), type 2 diabetes, type 1 diabetes, dyslipidemia and obesity.

19. A compound according to claim 1 for use as a medicament.

20. A pharmaceutical composition comprising at least one compound according to claim 1 together with one or more pharmaceutically acceptable carriers or excipients.

21. A pharmaceutical composition according to claim 20 in unit dosage form, said composition comprising from about 0.05 mg to about 1000 mg of said compound.

22. Use of a compound according to any one of the claims 1 to 18 for the preparation of a medicament for the treatment of disorders or diseases, wherein a glucagon antagonistic action is beneficial.

23. Use of a compound according to any one of the claims 1 to 18 for the preparation of a medicament for the treatment of glucagon-mediated disorders and diseases.

24. Use of a compound according to any one of the claims 1 to 18 for the preparation of a medicament for the treatment of hyperglycemia.

25. Use of a compound according to any one of the claims 1 to 18 for the preparation of a medicament for lowering blood glucose in a mammal.

26. Use of a compound according to any one of the claims 1 to 18 for the preparation of a medicament for the treatment of IGT.

27. Use of a compound according to any one of the claims 1 to 18 for the preparation of a medicament for the treatment of type 2 diabetes.

28. Use according to claim 27 for the preparation of a medicament for the delaying or prevention of the progression from IGT to type 2 diabetes.

29. Use according to claim 27 for the preparation of a medicament for the delaying or prevention of the progression from non-insulin requiring type 2 diabetes to insulin requiring type 2 diabetes.

30. Use of a compound according to any one of the claims 1 to 18 for the preparation of a medicament for the treatment of type 1 diabetes.

31. Use of a compound according to any one of the claims 1 to 18 for the preparation of a medicament for the treatment of obesity.

32. Use of a compound according to any one of the claims 1 to 18 for the preparation of a medicament for the treatment of dyslipidemia.

33. Use according to any one of the claims 22 to 32 in a regimen which comprises treatment with a further antidiabetic agent.

34. Use according to any one of the claims 22 to 33 in a regimen which comprises treatment with a further antiobesity agent.

35. Use according to any one of the claims 22 to 34 in a regimen which additionally comprises treatment with a further antihyperlipidemic agent.

36. Use according to any one of the claims 22 to 35 in a regimen which additionally comprises treatment with an antihypertensive agent.

37. A method for treating disorders or diseases wherein a glucagon antagonistic action is beneficial, said method comprising administering to a subject in need thereof an effective amount of a compound according to claim 1.

38. The method according to claim 37, wherein the effective amount of the compound is in the range of from about 0.05 mg to about 2000 mg per day.

39. A compound according to claim 16, wherein said compound has an IC₅₀ value of less than 500 nM as determined by Glucagon Binding Assay (I) or Glucagon Binding Assay (II).

40. A compound according to claim 16, wherein said compound has an IC₅₀ value of less than 100 nM as determined by Glucagon Binding Assay (I) or Glucagon Binding Assay (II).

41. A pharmaceutical composition according to claim 20 in unit dosage form, said composition comprising from about 0.1 mg to about 500 mg of said compound.

42. A pharmaceutical composition according to claim 20 in unit dosage form, said composition comprising from about 0.5 mg to about 200 mg of said compound.

43. The method according to claim 37, wherein the effective amount of the compound is in the range of from about 0.1 mg to about 1000 mg per day.

44. The method according to claim 37, wherein the effective amount of the compound is in the range of from about 0.5 mg to about 500 mg per day.

45. A method for treating hyperglycemia, said method comprising administering to a subject in need thereof an effective amount of a compound according to claim 1.

46. A method for lowering blood glucose in a mammal, said method comprising administering to said mammal an effective amount of a compound according to claim 1.

47. A method for treating impaired glucose tolerance (IGT), said method comprising administering to a subject in need thereof an effective amount of a compound according to claim 1.

48. A method for treating type 2 diabetes, said method comprising administering to a subject in need thereof an effective amount of a compound according to claim 1.

49. A method for delaying or preventing progression from impaired glucose tolerance (IGT) to type 2 diabetes, said method comprising administering to a subject in need thereof an effective amount of a compound according to claim 1.

50. A method for delaying or preventing progression from non-insulin requiring type 2 diabetes to insulin requiring type 2 diabetes, said method comprising administering to a subject in need thereof an effective amount of a compound according to claim 1.

51. A method for treating type 1 diabetes, said method comprising administering to a subject in need thereof an effective amount of a compound according to claim 1.

52. A method for treating obesity, said method comprising administering to a subject in need thereof an effective amount of a compound according to claim 1.

53. A method for treating dyslipidemia, said method comprising administering to a subject in need thereof an effective amount of a compound according to claim 1.

54. The method according to claim 37, said method further comprising treatment with an antidiabetic agent.

55. The method according to claim 37, said method further comprising treatment with an antiobesity agent.

56. The method according to claim 37, said method further comprising treatment with an antihyperlipidemic agent.

57. The method according to claim 37, said method further comprising treatment with an antihypertensive agent.
